SEQUENCE LISTING (52 sequences)

SEQ ID NO: 1 - RNA, 34 nt, Artificial Sequence
  34-nucleotide stretch (nucleotides 14653 - 14686) in ORF7 of Lelystad virus
auggccagccagucaaucaacugugccaguugcu

SEQ ID NO: 2 - RNA, 41 nt, Artificial Sequence
  hairpin within the 3′ UTR of Lelystad virus
aggugaauggccgcgauuggcguguggccucugagucaccu

SEQ ID NO: 3 - DNA, 34 bp, Artificial Sequence
  34-nucleotide stretch (nucleotides 14653 - 14686) in ORF7 of Lelystad virus
atggccagccagtcaatcaactgtgccagttgct

SEQ ID NO: 4 - protein, 128 aa, Porcine reproductive and respiratory syndrome virus
  SITE (1)..(128): N protein
MAGKNQSQKKKKSTAPMGNGQPVNQLCQLLGAMIKSQRQQPRGGQAKKKKPEKPHFPLAAEDDIRHHLTQTERSLCLQSIQTAFNQGAGTASLSSSGKVSFQVEFMLPVAHTVRLIRVTSTSASQGAS

SEQ ID NO: 5 - protein, 123 aa, Porcine reproductive and respiratory syndrome virus
  SITE (1)..(123): N protein
MPNNNGKQQKRKKGDGQPVNQLCQMLGKIIAQQNQSRGKGPGKKNKKKNPEKPHFPLATEDDVRHHFTPSERQLCLSSIQTAFNQGAGTCTLSDSGRISYTVEFSLPTHHTVRLIRVTASPSA

SEQ ID NO: 6 - DNA, 122 bp, Porcine reproductive and respiratory syndrome virus
  misc_feature (1)..(122): 3′ UTR
ttaaacagtcaggtgaatggccgcgattggcgtgtggcctctgagtcacctattcaattagggcgatcacatgggggtcatacttaatcaggcaggaaccatgtgaccgaaattaaaaaaaa

SEQ ID NO: 7 - DNA, 159 bp, Porcine reproductive and respiratory syndrome virus
  misc_feature (1)..(159): 3′ UTR
tgggctggcattcttgaggcatctcagtgtttgaattggaagaatgtgtggtgaatggcactgattgacattgtgcctctaagtcacctattcaattagggcgaccgtgtgggggtgagatttaattggcgagaaccatgcggccgaaattaaaaaaaa

SEQ ID NO: 8 - DNA, 18 bp, Artificial Sequence
  Primer 119R218R
atgacatccggcaccacc

SEQ ID NO: 9 - DNA, 21 bp, Artificial Sequence
  Primer LV20
cctgattaaaagcttgacccc

SEQ ID NO: 10 - DNA, 21 bp, Artificial Sequence
  Primer LV75
tctaggaattctagacgatcg

SEQ ID NO: 11 - DNA, 47 bp, Artificial Sequence
  Primer LV155
acgtgcgttaacctcgtcaagtatggccggtaaaaaccagagccaga

SEQ ID NO: 12 - DNA, 34 bp, Artificial Sequence
  Primer LV204
acgtgcttaattaaccttgactggcggatgtaga

SEQ ID NO: 13 - DNA, 29 bp, Artificial Sequence
  Primer LV213
tgcaagttaattaaggtgaatggccgcga

SEQ ID NO: 14 - DNA, 26 bp, Artificial Sequence
  Primer LV214
gactgtttaattaactggcggatgta

SEQ ID NO: 15 - DNA, 26 bp, Artificial Sequence
  Primer LV215
gactgtttaattaagtcacgcgaatc

SEQ ID NO: 16 - DNA, 26 bp, Artificial Sequence
  Primer LV239
tgcaagttaattaagcctctgagtca

SEQ ID NO: 17 - DNA, 25 bp, Artificial Sequence
  Primer LV263
gactgtttaattaagcggatgtaga

SEQ ID NO: 18 - DNA, 25 bp, Artificial Sequence
  Primer LV264
gactgttaattaagatgtagaagtc

SEQ ID NO: 19 - DNA, 25 bp, Artificial Sequence
  Primer LV265
gactgttaattaagtagaagtcacg

SEQ ID NO: 20 - DNA, 25 bp, Artificial Sequence
  Primer LV266
gactgttaattaagaagtcacgcga

SEQ ID NO: 21 - DNA, 20 bp, Artificial Sequence
  Primer 118U250
cagccaggggaaaatgtggc

SEQ ID NO: 22 - DNA, 20 bp, Artificial Sequence
  Primer 12U94R
cacctgtacctgctcattgt

SEQ ID NO: 23 - DNA, 20 bp, Artificial Sequence
  Primer 25U101
gttctagcccaacaggtatc

SEQ ID NO: 24 - DNA, 23 bp, Artificial Sequence
  Primer LV2
agcgggaaggatccaccgagtat

SEQ ID NO: 25 - DNA, 20 bp, Artificial Sequence
  Primer LV17
cccttgacgagctcttcggc

SEQ ID NO: 26 - DNA, 61 bp, Artificial Sequence
  Primer LV76
tctaggaattctagacgatcgtttttttttttttttttttttttttttttttttttttttt

SEQ ID NO: 27 - DNA, 20 bp, Artificial Sequence
  Primer LV78
ccctgggatgaatctatggt

SEQ ID NO: 28 - DNA, 22 bp, Artificial Sequence
  Primer LV79
gacaagatcatcagagtatacc

SEQ ID NO: 29 - DNA, 20 bp, Artificial Sequence
  Primer LV84
agagcttcaggacactgacc

SEQ ID NO: 30 - DNA, 36 bp, Artificial Sequence
  Primer LV112
ccattcacctgactgtttaattaacttgcaccctga

SEQ ID NO: 31 - DNA, 20 bp, Artificial Sequence
  Primer LV118
ttaccacctactctccaccg

SEQ ID NO: 32 - DNA, 21 bp, Artificial Sequence
  Primer LV132
cctactgtgccctatagtgtc

SEQ ID NO: 33 - DNA, 44 bp, Artificial Sequence
  Primer LV151
accagagccagaagaaaaagaaaagtacagctgggtgcaatgat

SEQ ID NO: 34 - DNA, 44 bp, Artificial Sequence
  Primer LV152
accagagccagaagaaaaagaaaagtacagctgccagttgctgg

SEQ ID NO: 35 - DNA, 44 bp, Artificial Sequence
  Primer LV153
accagagccagaagaaaaagaaaagtacagcttcaatcaactgt

SEQ ID NO: 36 - DNA, 44 bp, Artificial Sequence
  Primer LV154
accagagccagaagaaaaagaaaagtacagctatggccagccag

SEQ ID NO: 37 - DNA, 35 bp, Artificial Sequence
  Primer LV188
acgtgcgttaactaaggtgcaatgataaagtccca

SEQ ID NO: 38 - DNA, 36 bp, Artificial Sequence
  Primer LV189
acgtgcgttaactaaatcccggcaccacctcaccca

SEQ ID NO: 39 - DNA, 35 bp, Artificial Sequence
  Primer LV190
acgtgcgttaactaagggaaggtcagttttcaggt

SEQ ID NO: 40 - DNA, 35 bp, Artificial Sequence
  Primer LV191
acgtgcgttaactaacgcctgattcgcgtgacttc

SEQ ID NO: 41 - DNA, 33 bp, Artificial Sequence
  Primer LV195
acgtgcgttaactaaccgatggggaatggccag

SEQ ID NO: 42 - DNA, 42 bp, Artificial Sequence
  Primer LV196
ggagtggttaacctcgtcaagtaaccgatggggaatggccag

SEQ ID NO: 43 - DNA, 31 bp, Artificial Sequence
  Primer LV197
acgtgcgttaacggccggtaaaaaccagagc

SEQ ID NO: 44 - DNA, 30 bp, Artificial Sequence
  Primer LV198
gctcgtgctagcctttagcatcacatacac

SEQ ID NO: 45 - DNA, 34 bp, Artificial Sequence
  Primer LV200
acgtgcttaattaacccagcaactggcacagttg

SEQ ID NO: 46 - DNA, 34 bp, Artificial Sequence
  Primer LV201
acgtgcttaattaaatgtcatcttcagcagccag

SEQ ID NO: 47 - DNA, 34 bp, Artificial Sequence
  Primer LV202
acgtgcttaattaaccgctggatgaaagcgacgc

SEQ ID NO: 48 - DNA, 34 bp, Artificial Sequence
  Primer LV203
acgtgcttaattaacgcactgtatgagcaaccgg

SEQ ID NO: 49 - DNA, 54 bp, Artificial Sequence
  Primer LV216
accagagccagaagaaaaagaaaagtacagctccgatggggagggtgcaatgat

SEQ ID NO: 50 - DNA, 42 bp, Artificial Sequence
  Primer LV268
accagagccagaagaaaaagaaaagtacagctccgatgggga

SEQ ID NO: 51 - DNA, 42 bp, Artificial Sequence
  Primer LV269
ctccgatggggaatggccagccagtgttagaactgtgccagt

SEQ ID NO: 52 - DNA, 51 bp, Artificial Sequence
  Primer LV270
tgcaagttaattaaacagtcaggtgaatggccgcctaacgcgtgtggcctc

(19) United States
(12) Patent Application Publication
(10) Pub. No.: US 2004/0213805 A1
(43) Pub. Date: Oct. 28, 2004

(54) Deletions in arterivirus replicons

(76) Inventor: Monique Helene Verheije, JC Dronten (NL)

Correspondence Address: TRASK BRITT, P.O. BOX 2550, SALT LAKE CITY, UT, 84110, US

(21) Appl. No.: 10/719,895

(22) Filed: Nov. 21, 2003

Related U.S. Application Data
(63) Continuation of application No. PCT/NL02/00314, filed on May 16, 2002.
(63) Continuation of application No. 09/874,626, filed on Jun. 5, 2001.
Application No. 09/874,626 is a continuation of application No. 09/297,535, filed on Oct. 12, 1999, now Pat. No. 6,268,199.

(30) Foreign Application Priority Data
May 21, 2001 (EP) 01201921.2

(51) Int. Cl. A61K 39/12; C12N 7/00

(52) U.S. Cl. 424/199.1; 435/235.1

(57) ABSTRACT

The invention relates to recombinant Arterivirus replicons. The invention provides the insight that an Arterivirus replicon having at least some of its original arteriviral nucleic acid encoding ORF-7 deleted, as provided herein, can still be capable of in vivo RNA replication, even when further comprising nucleic acid derived from at least one heterologous micro-organism, thereby also providing viable Arteriviruses with deletions proximal to the 3′ end of the genome.

### CROSS-REFERENCE TO RELATED APPLICATIONS

This application is a continuation of International Application No. PCT/NL02/00314, filed on 16 May 2002, which was published in English on Nov. 28, 2002, as International Publication No. WO 02/095040, designating the United States of America, and claims the benefit of U.S. application Ser. No. 09/874,626, filed Jun. 5, 2001, which is a continuation of U.S. application Ser. No. 09/297,535, filed Oct. 12, 1999, now U.S. Pat. No. 6,628,199, the entire content of each of which is hereby incorporated by this reference.

### TECHNICAL FIELD

The invention relates to recombinant Arterivirus replicons.

### BACKGROUND

Porcine reproductive and respiratory syndrome virus (PRRSV) is a positive strand RNA virus that belongs to the Arteriviridae family (reviewed in Snijder and Meulenberg, 1998), together with equine arteritis virus (EAV), lactate dehydrogenase-elevating virus (LDV), and simian hemorrhagic fever virus (SHFV) (Meulenberg et al., 1993b). On the basis of their similar genomic organization and replication strategy, the arteriviruses have been grouped into the new order of Nidovirales together with the coronaviruses and the toroviruses (Cavanagh, 1997). PRRSV is the causative agent of respiratory problems in pigs and stillbirths in sows, and accounts for huge economical losses worldwide. PRRSV was first isolated in the Netherlands in 1991 (Wensvoort et al., 1991), and was designated Lelystad virus (LV).

At present, over 100 isolates of PRRSV have been identified, mainly from Europe and North America. The genome of PRRSV is a 5′-capped and 3′-polyadenylated RNA molecule of 15.1 kb (Meulenberg et al., 1993b). The 5′ two-third of this RNA is translated into two large polyproteins. These are subsequently cleaved by virus-encoded proteases to yield at least 12 non-structural proteins, including the viral RdRp (Snijder et al., 1994; van Dinten et al., 1999; van Dinten et al., 1996; van Marle et al., 1999b; Wassenaar et al., 1997). In addition, a set of subgenomic (sg) mRNAs is produced through a process of discontinuous mRNA transcription. These sg mRNAs each contain a leader sequence derived from the 5′ UTR fused to a body part derived from the 3′ part of the genome (de Vries et al., 1990; Lai, 1990; Meulenberg et al., 1995; Meulenberg et al., 1993a). Leader-body fusion occurs at a transcription-regulating sequence (TRS) and results in the production of a 3′ nested set of sg mRNAs. They collectively specify the viral structural proteins.

The process of discontinuous transcription has not been resolved conclusively and may occur during plus or minus strand synthesis (reviewed in (Lai et al., 1994; Sawicki and Sawicki, 1998; van Marle et al., 1995)). From each sg mRNA, only the 5′ most ORF is thought to be translated. ORF7 encodes the nucleocapsid protein N, ORF6 the membrane protein M, ORF5 the major envelope glycoprotein GP5, and

The invention relates to recombinant Arterivirus replicons.

Porcine reproductive and respiratory syndrome virus (PRRSV) is a positive strand RNA virus that belongs to the Arteriviridae family (reviewed in Snijder and Meulenberg, 1998), together with equine arteritis virus (EAV), lactate dehydrogenase-elevating virus (LDV), and simian hemorrhagic fever virus (SHFV) (Meulenberg et al., 1993b). On the basis of their similar genomic organization and replication strategy, the arteriviruses have been grouped into the new order of Nidovirales together with the coronaviruses and the toroviruses (Cavanagh, 1997). PRRSV is the causative agent of respiratory problems in pigs and stillbirths in sows, and accounts for huge economical losses worldwide. PRRSV was first isolated in the Netherlands in 1991 (Wensvoort et al., 1991), and was designated Lelystad virus (LV).

At present, over 100 isolates of PRRSV have been identified, mainly from Europe and North-America. The genome of PRRSV is a 5′-capped and 3′-polyadenylated RNA molecule of 15.1 kb (Meulenberg et al., 1993b). The 5′ two-third of this RNA is translated into two large polyproteins. These are subsequently cleaved by virus-encoded proteases to yield at least 12 non-structural proteins, including the viral RdRp (Snijder et al., 1994; van Dinten et al., 1999; van Dinten et al., 1996; van Marle et al., 1999b; Wassenaar et al., 1997). In addition, a set of subgenomic (sg) mRNAs is produced through a process of discontinuous mRNA transcription. These sg mRNAs each contain a leader sequence derived from the 5′ UTR fused to a body part derived from the 3′ part of the genome (de Vries et al., 1990; Lai, 1990; Meulenberg et al., 1995; Meulenberg et al., 1993a). Leader-body fusion occurs at a transcription-regulating sequence (TRS) and results in the production of a 3′ nested set of sg mRNAs. They collectively specify the viral structural proteins.

The process of discontinuous transcription has not been resolved conclusively and may occur during plus or minus strand synthesis (reviewed in (Lai et al., 1994; Sawicki and Sawicki, 1998; van Marle et al., 1995)). From each sg mRNA, only the 5′ most ORF is thought to be translated. ORF7 encodes the nucleocapsid protein N, ORF6 the membrane protein M, ORF5 the major envelope glycoprotein GP5, and ORFs2-4 the minor envelope glycoproteins GP2, GP3, and GP4 (Meulenberg et al., 1995). Recently, a novel structural protein called E was described for EAV (Snijder et al., 1999), which is also translated from sg mRNA2 in addition to GP2. Besides the coding regions, the PRRSV genome contains a 5′UTR of 221 nucleotides (Snijder and Meulenberg, 1998), which carries the cap at its 5′ end (Meulenberg et al., 1998; Sagripanti et al., 1986), and a 3′UTR of 114 nucleotides to which the poly(A)-tail is attached (Meulenberg et al., 1993b).

Positive strand RNA viruses replicate in infected cells by a process which is mediated by RNA-dependent RNA-polymerase (RdRp). In this process, the positive strand genomic RNA serves as a template for the production of negative strand genomic RNA, which is used in turn as a template for the synthesis of new plus strands. The process of replication requires the recruitment of the RdRp to specific sequences or structures within the templates, also known as cis-acting elements. These elements are usually located in the non-coding regions at the termini of the viral RNA, where RdRp complexes initiate the synthesis of plus and minus strands (Buck, 1996). Cis-acting elements have been characterized for several viruses and show a wide variety of structures. They can be structures with no apparent structure, e.g. the plus strand promoter from a satellite RNA of a carnovirus (Guan et al., 2000); stem-loop structures, e.g. in the 5′ untranslated region (UTR) of arterivirus RNAs (Hwang and Brinton, 1998), pseudoknots, e.g. in the 3′UTR of coronavirus RNAs (Williams et al., 1995) or of alfamo- and ilarvirus RNAs (Olsthoorn et al., 1999), tRNA-like structures at the ends of several plant viral RNAs (Dreher, 1999), or a kissing loop interaction as found in the 3′UTR of enteroviral RNA (Melchers et al., 2000). In a few cases, cis-acting elements are located within a coding region, e.g. the long-range pseudoknot of bacteriophage Qb RNA (Klovins and van Duin, 1999). This is the only known sequence in the coding region which is involved in a long-range interaction that is essential for RNA replication.

Little is known about the requirements for arterivirus RNA replication and transcription. Regions of both coding and non-coding sequences may be involved in these processes. Cis-acting elements for EAV genome replication, transcription, and packaging have been roughly mapped by using a Defective Interfering (DI) genome (Molenkamp et al., 2000a). So far, it has not been elucidated which sequences in the region of the arterivirus genome encoding the structural proteins are essential for RNA replication and/ or transcription.

The invention provides the insight that an Arterivirus replicon having at least some of its original arteriviral nucleic acid encoding ORF-7 deleted, as provided herein, can still be capable of in vivo RNA replication, even when further comprising nucleic acid derived from at least one heterologous micro-organism, thereby also providing viable Arteriviruses with deletions proximal to the 3′ end of the genome.

The present application describes the requirements for replication and transcription on the RNA/nucleic acid level, and the use for vaccine development or vector systems. Further this application teaches how these biological processes can be influenced. It demonstrates that for producing a replicon, it is essential that a long distance interaction between a (34-)nucleotide stretch in a coding region of the viral genome (which stretch is highly conserved among PRRSV isolates and folds into a putative stem-loop structure) and particularly between a 7-base sequence within the loop of this structure needs be maintained with a sequence present in the 3′ noncoding region, which in turn occurs in the loop of a predicted, strongly conserved hairpin structure. However, it is the base-pairing ability, not the sequences per se, that is essential, for example, complementary substitution of a short (3-7, preferably 5-)base sequence in either of the loops still allows the generation of a replicon.

The invention relates to recombinant Arterivirus replicons and methods to obtain these. The invention provides the insight that an Arterivirus replicon having at least some of its original arteriviral nucleic acid (such as encoding a distinct part of ORF-7) deleted, as provided herein, can still be capable of in vivo RNA replication, even when further comprising nucleic acid derived from at least one heterologous micro-organism, thereby also providing viable Arteriviruses with deletions proximal to the 3′end of the genome, provided that said long-distance interaction is kept in place. To obtain further Arterivirus replicons, the invention provides a method for generating a replicon wherein a short, approximately 5-base sequence in either of the loops is modified in that it is complementary substituted while maintaining said long-distance interaction, as for example shown in FIG. 4. As said, the invention provides a method for generating a replicon of an Arterivirus, preferably of PRRSV, wherein by mutation the genome of said Arterivirus is altered, but wherein the ability of the two predicted loops to base-pair, (albeit not their primary sequences per se) is functionally kept intact. The results show that the kissing loop interaction that we observed stabilizes a three-dimensional conformation within the 3′ terminal region of the viral genome onto which an RNA polymerase complex assembles for the initiation of negative-strand RNA synthesis, allowing the replication to proceed. The invention furthermore provides arterivirus replicon having at least some of its original arteriviral nucleic acid (such as that encoding ORF-7) deleted, wherein the primary sequences of said loops are no longer wild-type sequences (as for example known from Genbank datasubmissions NC-001961, AF066183, AF331831, NC-002534, U87392, NC-002533, M96262, AF184212, NC-001639, AF159149, AF046869, and U15146) but wherein the ability of the two loops to base-pair (as for example identified in FIG. 4) is functionally kept intact.

This finding thus provides distinct metes and bounds for the production of Arterivirus replicons that were not known earlier, the invention thus provides elegant directions for steering around questions as to the regions of the viral genome in which deletions are tolerated which may have been raised but were not answered earlier. For example, Molenkamp et al. (JGV 81:2491,2000) have extensively studied the requirements on protein level for RNA replication and subgenomic transcription of EAV by point- and deletion mutations in all structural ORFs. Their conclusion was that structural proteins are not essential for replication and transcription, thereby not noting that at least one part of the coding region is essential for replication. Also Bramel-Verheije et al., (The VIII Int. Symp. on Nidoviruses, 20-25 May 2000) describes that small deletions in ORF-7 can be tolerated in reproduction of infectious PRRSV, here we show that to obtain replicons such deletions can even be much larger, provides said long-distance interaction is kept in place, information that can also not be gained from Genbank datasubmissions NC-001961, AF066183, AF331831, NC-002534, U87392, NC-002533, M96262, AF184212, NC-001639, AF159149, AF046869, and U15146 of nucleotide sequences of related viruses provide no information whatsoever about functions of sequences or regions important for RNA replication and subgenomic transcription.

Also, WO 005387 provides an infectious clones of PRRSV eventually supplemented with heterologous genetic material, but does not teach the minimally essential requirement of said long-distance interaction, nor do U.S. Pat. No. 6,110,467 (relating to a PRRS vaccine, attenuated or inactivated, obtained by serial passages of defined PRRSV strains belonging to the US genotype) nor WO 96/06619 which relatesto polynucleic acids and proteins originated from PRSV and there use. Nucleic acids are used for encoding one or more PRRSV proteins in expression systems such as baculovirus but no recombinant replicating system based on PRRSV is provided. U.S. Pat. No. 5,998,601 relates to a DNA sequence which comprises full length or part of VR2332, an US PRRSV isolate, but does not teach the minimally essential requirement of said long-distance interaction either.

In one embodiment, the invention provides a replicon wherein a kissing loop interaction between 3′ noncoding and coding sequences is maintained where its primary sequences essential for wild-type Arterivirus RNA replication are modified. Like many other positive strand RNA viruses, replication of arteriviruses requires cis-acting elements to initiate the synthesis of genomic negative strands. These cis-acting elements are now known to be located in the 5′ and 3′ non-coding regions, as well as in sequences from the long open reading frame 1ab (ORF1ab) encoding the nonstructural proteins. In this application, we provide evidence of the presence of cis-acting elements essential for replication in the region encoding the structural proteins of a porcine arterivirus. Deletions were introduced into the infectious cDNA clone of the Lelystad virus (LV) isolate of porcine reproductive and respiratory syndrome virus (PRRSV) and replication of these mutants was analyzed. We identified a stretch of 34 nucleotides (14653-14686) located within ORF7, which encodes the viral nucleocapsid (N) protein, to be essential for RNA replication. Strand-specific RT-PCR analysis of transcripts transfected into BHK-21 cells revealed that this region is required for negative strand genomic RNA synthesis. The 34-nucleotide stretch is highly conserved among PRRSV isolates and folds into a putative stem-loop structure. Interestingly, a 7-base sequence within the loop of this structure appeared to be complementary to a sequence present in the 3′ noncoding region, which in turn occurs in the loop of a predicted, strongly conserved hairpin structure. The suspected kissing loop interaction was confirmed by mutational analyses. Complementary substitution of a 5-base sequence in either of the loops abolished replication while the reciprocal exchange of the 5-base sequence between the two loops repaired the defect. Apparently, it is the base-pairing ability, not the sequences per se that is essential. A slight (44 nucleotides) upstream displacement of the 34-nucleotide domain rendered the viral RNA replication-negative. We conclude that the long-distance or predicted kissing interaction in the 3′ terminal region of the PRRSV genome stabilizes a higher-order conformation that allows the assembly of the replication complex required for the initiation of negative strand RNA synthesis. The effects of the deletions were tested by assaying the ability of these viral RNAs in cells as to the expression of the unaffected structural protein genes as well as by strand-specific RT-PCR. Our results identify a stretch of 34 nucleotides located within ORF7 as essential for viral RNA replication. These 34 nucleotides are predicted to form an RNA hairpin in which loop residues are complementary to nucleotides from the loop of another hairpin within the 3′UTR. In the detailed description, we provide further evidence that this so-called kissing loop interaction is required for RNA replication of PRRSV.

Furthermore, the invention provides the insight that an Arterivirus replicon having at least some of its original arteriviral nucleic acid encoding ORF-7 deleted, as provided herein, can still be capable of in vivo RNA replication, even when further comprising nucleic acid derived from at least one heterologous micro-organism. In another embodiment, the invention provides a deletion in the region around the N gene stop codon. Alignment of the N protein sequence and the 3′UTR of different PRRSV strains revealed heterogeneity at the C-terminus of the N protein and at the 5′ end of the 3′UTR. A deletion analysis of this region was therefore performed using the available infectious cDNA clone (Meulenberg et al., 1998a) of Lelystad virus (LV) to determine the limits of the sequences that can be removed without significantly affecting virus viability, hereby providing the generation of viable arterivirus mutants containing a deletion in the viral genome, which is stably maintained after multiple passages in vitro. The thus obtained attenuated live vaccine candidates of porcine reproductive and respiratory syndrome virus (PRRSV), each comprise one of a series of deletions introduced at the 3′ end of the viral genome, for example using the infectious cDNA clone of the Lelystad Virus (LV) isolate. RNA transcripts from the full-length cDNA clones were transfected into BHK-21 cells. The culture supernatant of these cells was subsequently used to infect porcine alveolar macrophages to detect the production of progeny virus. We show that C-terminal truncation of the nucleocapsid protein N, encoded by ORF7, was tolerated for up to 6 amino acids without blocking the production of infectious virus. Mutants containing larger deletions produced neither virus nor virus-like particles containing viral RNA. Deletion analysis of the 3′UTR immediately downstream of ORF7 showed that infectious virus was still produced after removal of 7 nucleotides behind the stop codon of ORF7. Deletion of 32 nucleotides in this region abolished RNA replication and, consequently, no infectious virus was formed. Serial passage on porcine alveolar macrophages demonstrated that the viable deletion mutants were genetically stable at the site of mutation. In addition, the deletions did not affect the growth properties of the recombinant PRRS viruses in vitro, while their antigenic profiles were similar to that of wild type virus. Immunoprecipitation experiments with the 6-residue N protein deletion mutant confirmed that the truncated protein was indeed smaller than the wild type N protein. The deletion mutants produced herein are excellent opportunities to prevent PRRS disease in pigs. For vaccine purposes of course we provide here only the generation of viable deletion mutants of PRRSV, hereby steering around basic issues as to the regions of the viral genome in which deletions are tolerated. In this respect, two considerations are important. First, PRRSV has a concise genome, like other RNA viruses. Since RNA viruses have evolved to optimal fitness, most of the genetic information is expected to be essential. Second, the ORFs that encode the structural proteins of the virus are partially overlapping. Deletions in overlapping regions would therefore result in the mutation of two structural proteins, which would almost inevitably lead to the production of a nonviable virus. Earlier studies showed that deletions in many conserved regions were lethal, contrary to a replicon according to the invention that is at least equipped with a functional kissing loop interaction essential for said replication, and/or displays a C-terminally truncated ORF-7 polypeptide wherein said truncation is within the limits as provided herein and thus does not effect the production of viable virus, albeit attenuating. The invention furthermore provides the use of a replicon according to the invention for obtaining a vaccine, said vaccine preferably comprising such a replicon; however a killed vaccine or subunit vaccine based on using the replicon to produce the necessary antigenic mass is also provided. Such a vaccine can be used for vaccinating animals, preferably pigs susceptible to PRRSV infections.

### FIGURE LEGENDS

FIG. 1A/B

Design and analysis of the deletions mutants of the recombinant cDNA clones of PRRSV. Parts were deleted by using restriction sites present in the cDNAs (A) or by insertion of PCR-fragments produced by PCR-mutagenesis (B) into pABV437, a full-length cDNA clone containing a PacI-site directly downstream of ORF7 (Meulenberg et al., 1998). The outline of the constructs, the deleted nucleotides, the plasmid numbers, and the expression of the M and N protein are indicated. The boxes indicate the regions that are present, the lines indicate the regions that are deleted. Staining was performed by IPMA with M-(MAb126.3) and N-(MAb122.17) specific antibodies at 24 hours after transfection. Positive staining is indicated by +; no staining is indicated by −.

FIG. 2

RT-PCR strategy for (A) and results of (B) the detection of genomic positive strand RNA (1) and genomic negative strand RNA (2), sg positive strand mRNA7 (3) and sg negative-strand mRNA7 (4). BHK-21 cells were electroporated with RNA transcripts from pABV437, pABV668, and pABV696, and cellular RNA was isolated 12 hours after transfection. The viral RNA was reverse transcribed and amplified by PCR, as outlined in A. Products were analyzed in a 1% agarose gel. Numbers of the constructs from which the amplification products were derived are indicated beneath the lanes. The numbers on the left indicate the marker sizes in kilobases. The nucleotide positions of the primers are indicated between brackets beneath the primers.

FIG. 3

(A) Schematic representation of the predicted secondary structure in the 34-nucleotide stretch (nucleotides 14653-14686) in ORF7 of LV (GenBank M96262). (B) Predicted secondary structure of a hairpin within the 3′UTR, with 7 nucleotides in its loop complementary to 7 nucleotides in the predicted loop within the 34-nucleotide stretch. Nucleotide differences with other PRRSV strains are indicated alongside.

FIG. 4

(A) Kissing loop interaction between the hairpins predicted within the 34-nucleotide stretch and the 3′UTR. Nucleotides that are complementary between the loops of both hairpins are boxed. (B) Complementarity requirements in the loops of the predicted RNA hairpins within ORF7 and the 3′UTR. Mutated nucleotides are in thin typeface and in italic. RNA transcripts of the full-length cDNA clones were transfected into BHK-21 cells and the expression of the M protein (MAb 126.3) was analyzed 24 hours later by IPMA.

FIG. 5

Relocation of nucleotides 14653 to 14686 by PCR-mutagenesis to a position directly behind the stop codon of ORF6, ensuring that the sequence encoding the M protein remained intact (pABV697).

FIG. 6

(A) Amino acid alignment of the N protein of LV and of VR2332. (B) Nucleotide alignment of the 3′UTRs of LV and VR2332. LV and VR2332 are the prototypes of the European and American PRRSV strains, respectively. Underlined is the part of the PacI-site present in the 3′UTR. The conserved residues are indicated beneath the alignments with *.

FIG. 7

Design and analysis of the deletion mutants of the recombinant cDNA clones of PRRSV. Deletions were introduced by PCR-mutagenesis, and cloned into the full-length cDNA clone pABV437 (Meulenberg et al., 1998a). The constructs, the deleted nucleotides, the plasmid numbers, the observed expression of M and N protein, and the production of viable virus are indicated. The boxes indicate the present regions, the lines indicate the deleted regions. Expression of the viral proteins M and N was analysed by IPMA 24 hours after transfection of BHK-21 cells using MAb 126.3 and MAb 122.17, respectively. Positive staining is indicated by +; no staining is indicated by −. The bar above the constructs indicates the antigenic domains of the N protein.

FIG. 8

Growth curves in PAMs of wild type virus vABV437 and of mutant viruses vABV693 and vABV746. PAMs were infected in duplicate with passage 5 of the indicated viruses at a multiplicity of infection of 0.05 and virus was harvested at the indicated time points. Virus titres were determined by end point dilution on PAMs (Wensvoort et al., 1986).

FIG. 9

Analysis of the N protein expressed by the wild type virus vABV437, and by the 6-amino acid N protein deletion mutant, vABV746. Proteins were immunoprecipitated from lysates of PAMs infected with passage 5 of vABV437 and vABV746. Labelling was performed for 4 hours starting at 15 hours after infection. The immunoprecipitated proteins were analysed by electrophoresis by SDS-PAG in a 14% acrylamide gel. The molecular weight of the marker proteins is indicated on the left in kilodaltons (kDa).

FIG. 10

Analysis of the supernatant of BHK-21 cells transfected with pABV747 and pABV437. Fifteen hours after transfection, the cells were labelled for 24 hours using 75 μl (10.5 mCi/ml) Tran[35-S]-label. Particles in the supernatant were concentrated and fractionated as described in detail in Materials and Methods. The proteins in the fractions were analysed by electrophoresis in a 14% SDS-PAG (A). RNA was isolated from the fractions and analysed by RT-PCR, for which the primers flanked the region in which the deletion was introduced (B). The sizes of the marker are indicated on the left in kilo daltons (kDa) (A) and in base pairs (bp) (B). The lane indicated by + contains the positive PCR control.

FIG. 11=Table 1

Table 1

Sequences of the primers used to introduce deletions by PCR, primers used to sequence the introduced mutations, and primers used for the strand-specific RT-PCR assays.

FIG. 12=Table 2

Table 2

Sequences of the primers used to introduce deletions by PCR and to sequence the introduced mutations. The orientation of the primers (+ and − for sense and antisense, respectively) and the location of each primer with respect to the nucleotide sequence of LV (GenBank M96262) are indicated.

### DETAILED DESCRIPTION

### EXAMPLE 1

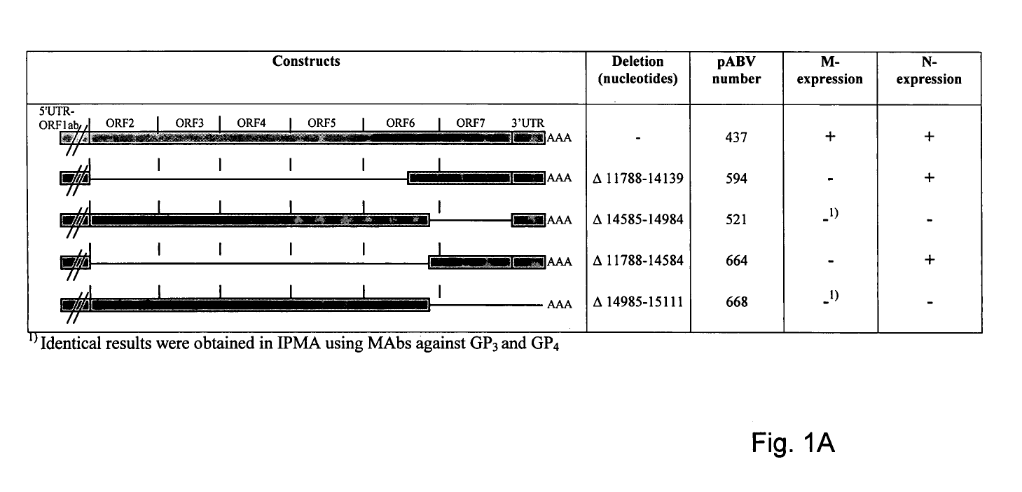

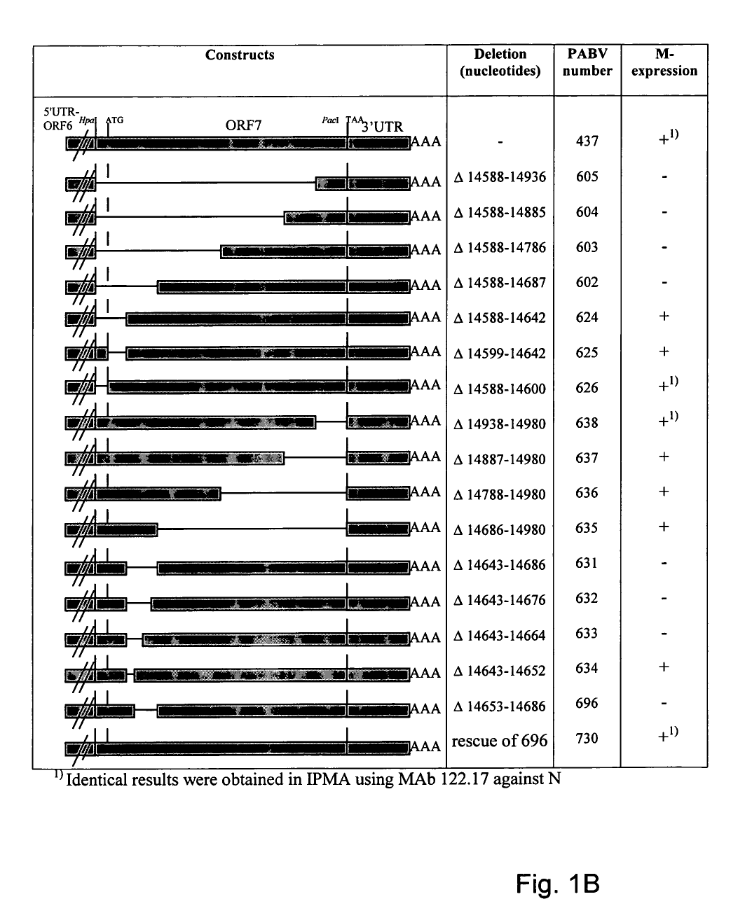

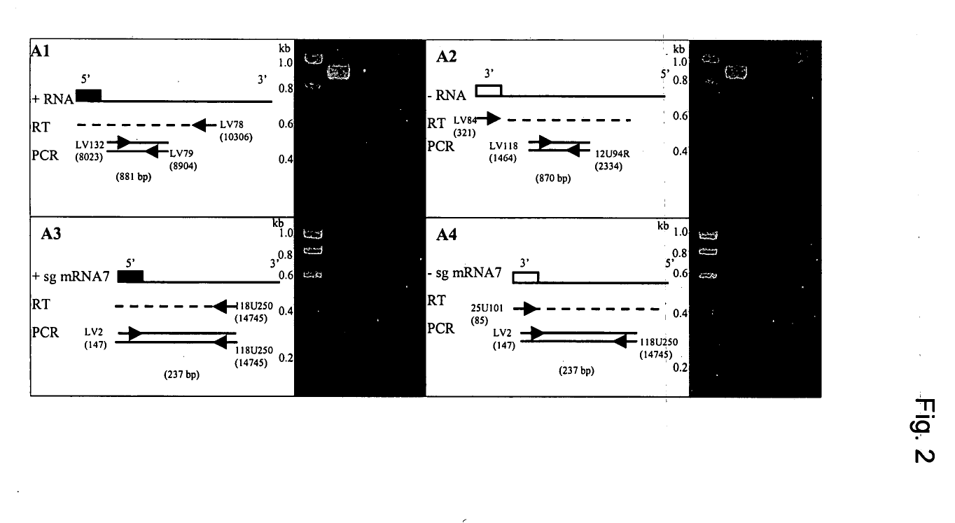

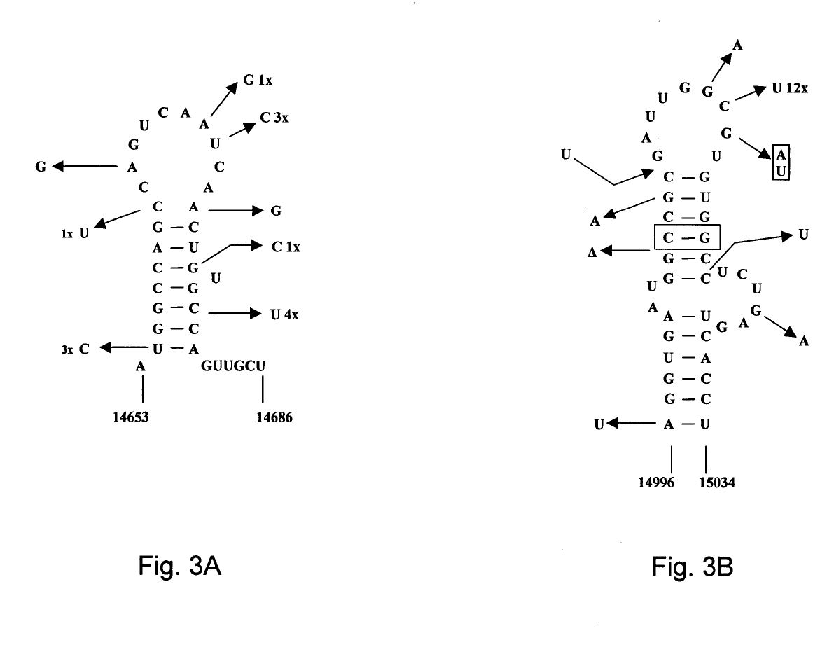

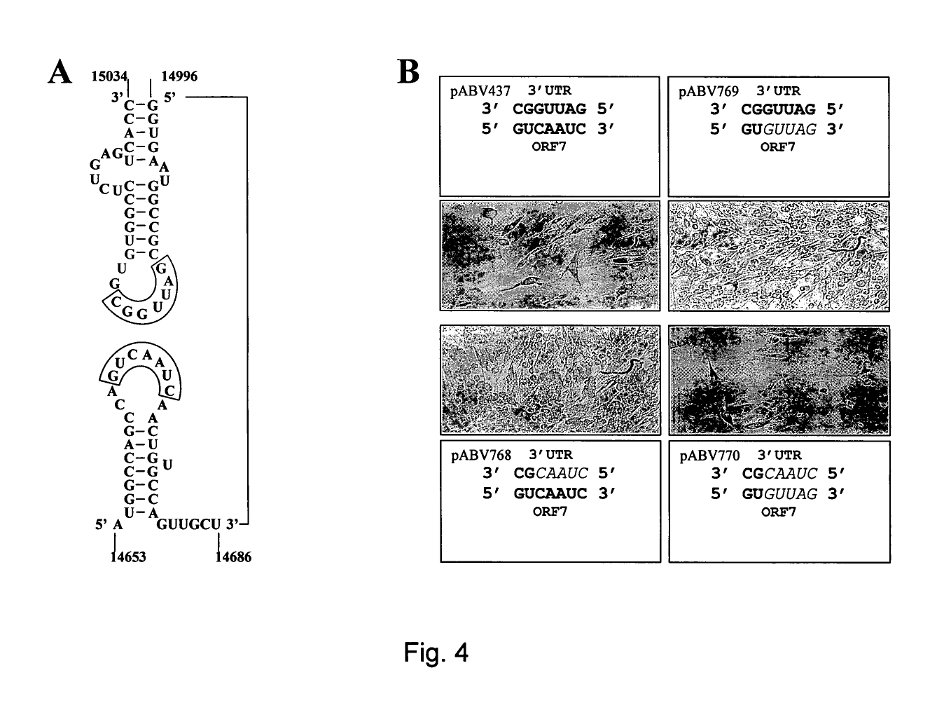

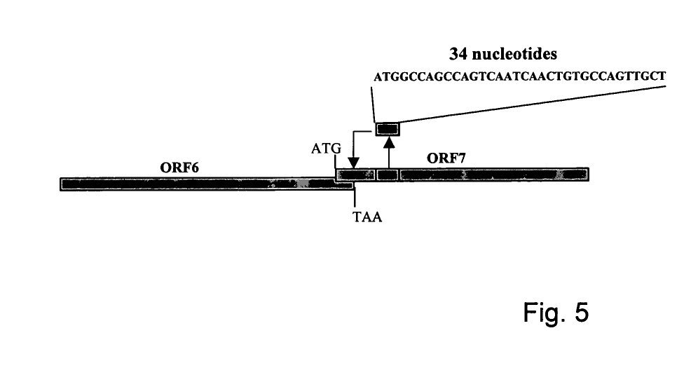

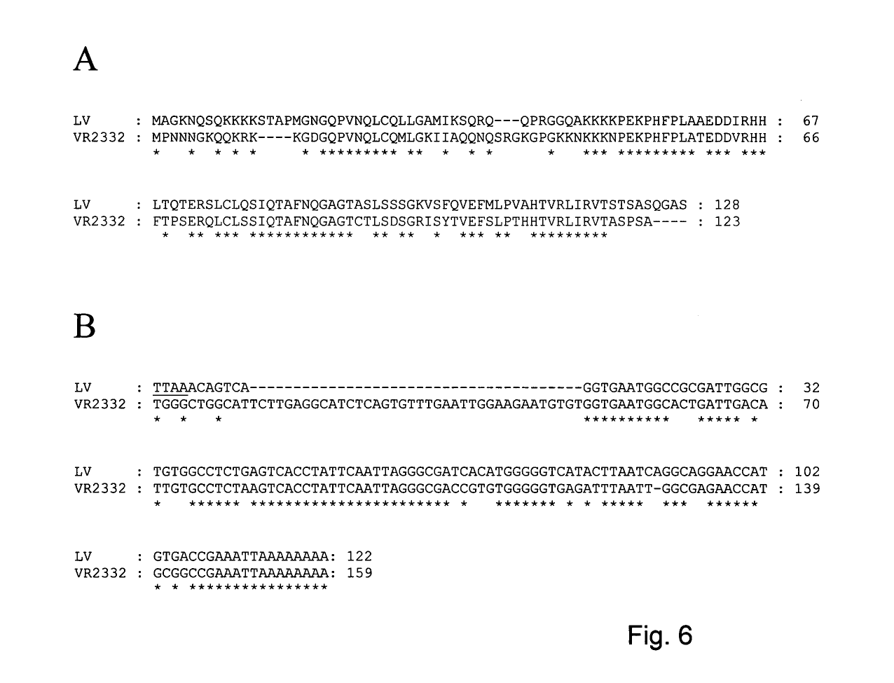

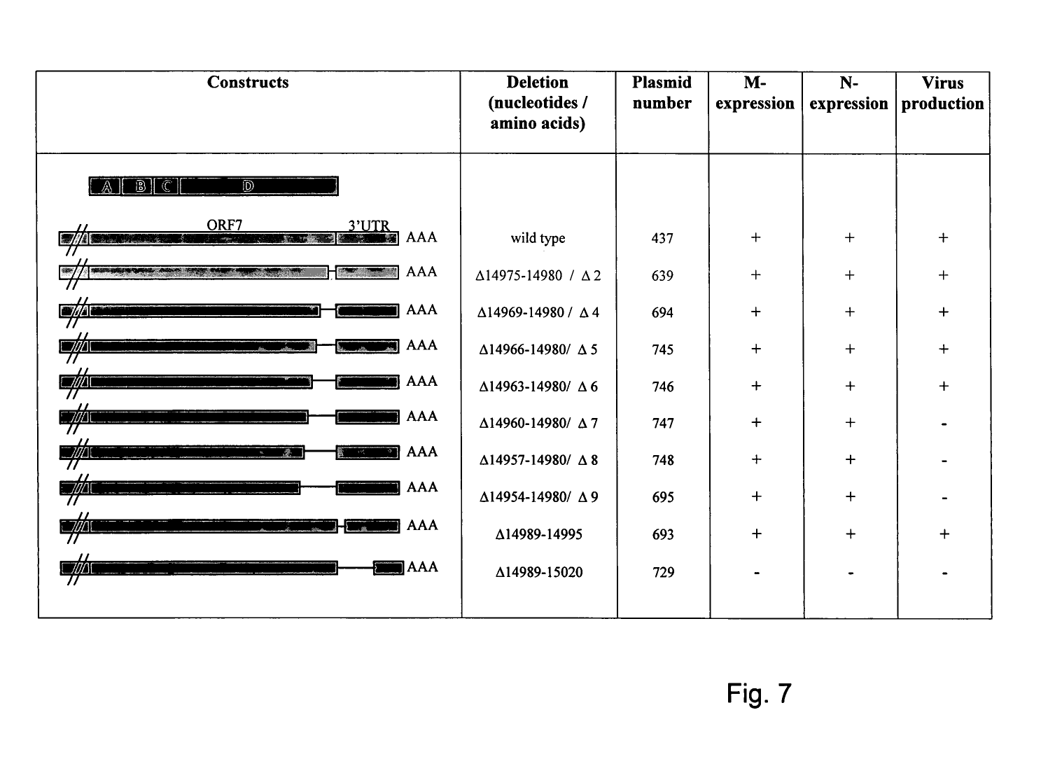

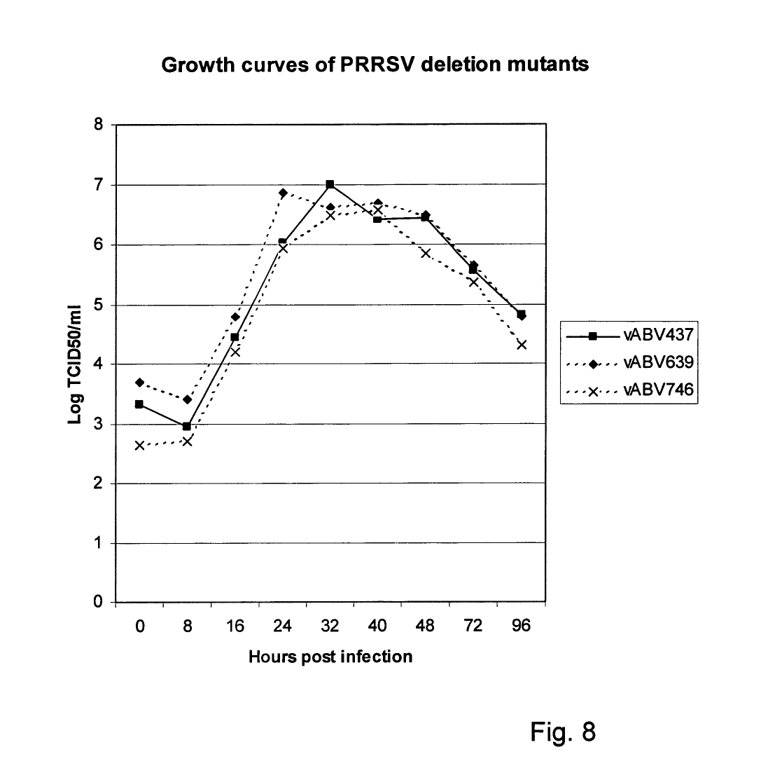

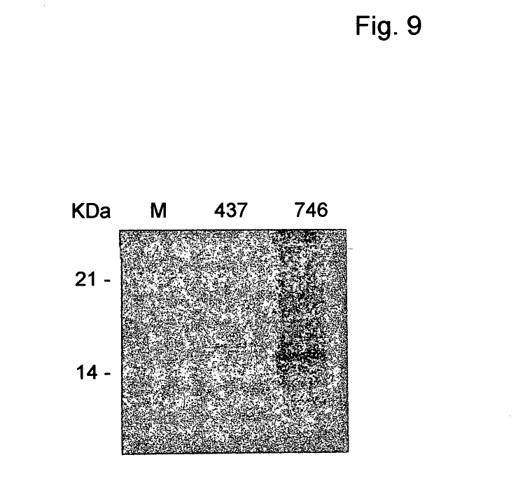

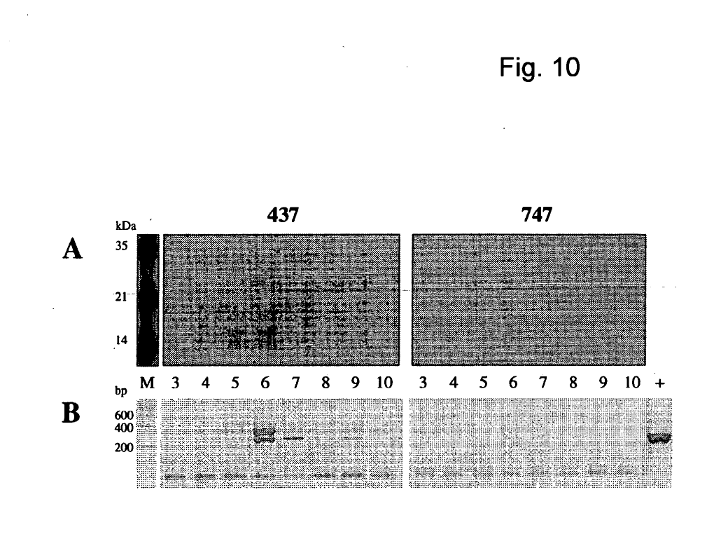

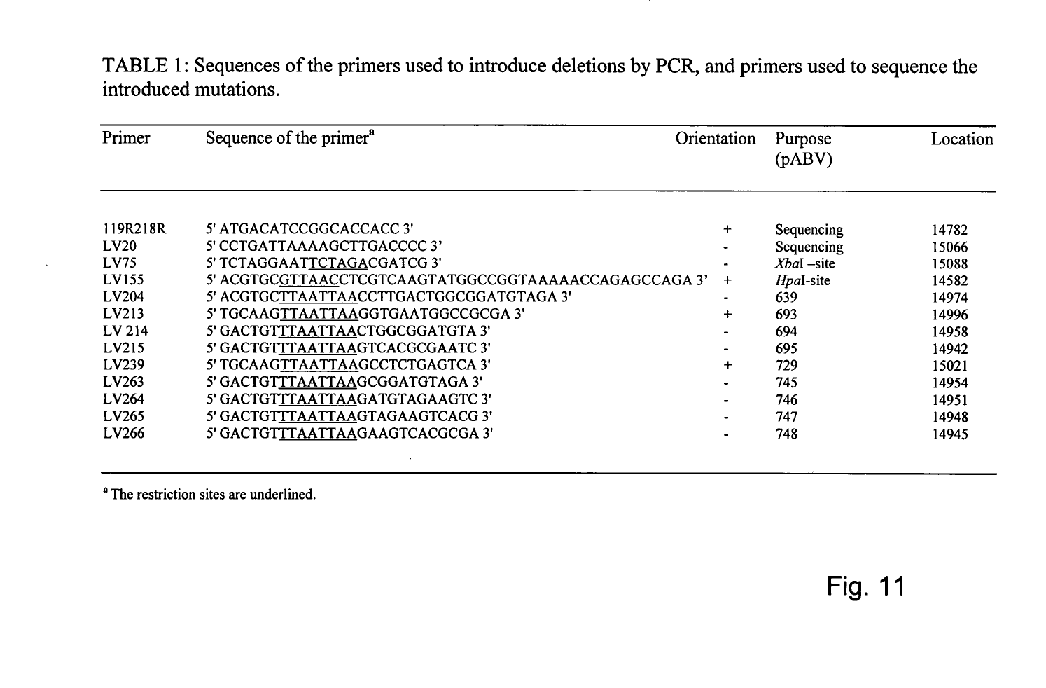

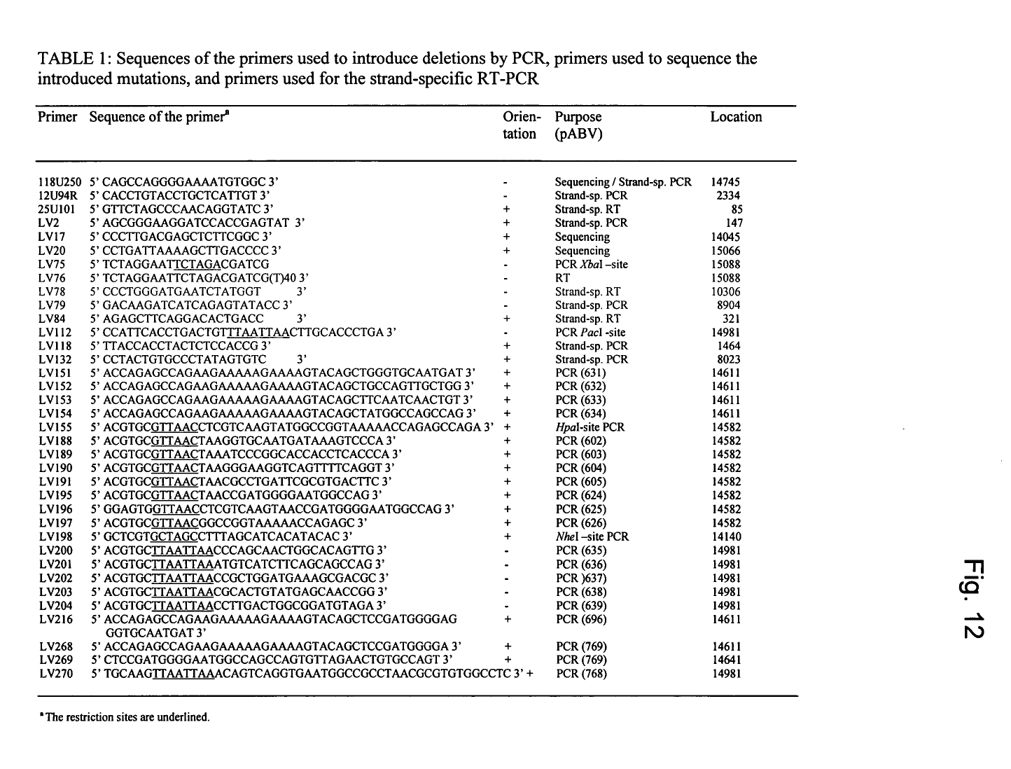

In order to elucidate whether genomic sequences encoding the structural proteins are essential for viral replication and/ or transcription, deletions were introduced into the infectious cDNA clone of LV (Meulenberg et al., 1998) and their RNA transcripts were transfected into BHK-21 cells. Their ability to transiently express the remaining viral structural protein genes was tested by immunoperoxidase monolayer assay (IPMA) 24 hours after transfection, as an indicator for replication and transcription. The design of the constructed cDNA clones and the results of the IPMA staining with monoclonal antibodies (MAbs) directed against the M and N proteins of transfected BHK-21 cells are compiled in FIG. 1A. RNA transcripts lacking ORF2 through the 5′ part of ORF6 (pABV594) induced the expression of the N protein in the transfected BHK-21 cells, indicating that sg mRNAs were still produced and that replication and transcription were not affected. In contrast, RNA transcripts lacking the entire ORF7 (pABV521) gene did not express any of the remaining structural proteins after transfection into BHK-21 cells. To find out whether sequences in the 3′ part of ORF6 caused the difference, we tested a construct from which again ORFs2 through ORF6, but now up to the TRS of ORF7 were deleted (pABV664). The RNA transcripts lacking this region were still able to induce N protein expression, albeit to a lower level, as indicated by a less intensive immunostaining (data not shown). The profound and specific effect of the removal of ORF7 indicated that this sequence is required either for genomic RNA replication or for sg mRNA transcription.

A 34 nucleotide stretch within ORF7 is essential for structural protein expression To locate more precisely the region(s) in ORF7 responsible for the observed effects, we constructed a collection of additional mutants that contained smaller deletions (FIG. 1B). Using PCR-mutagenesis, deletions increasing in length both at the 5′ end and at the 3′ end of ORF7 were introduced by into the infectious cDNA clone of LV. The RNA transcripts of these constructs were transfected into BHK-21 cells and tested for their ability to express PRRSV structural proteins by IPMA (FIG. 1B). We observed that large parts at the 3′ end of ORF7 (up to position 14689) could be deleted without affecting M protein expression, whereas small deletions from the 5′ end were already sufficient to inhibit M protein expression. A detailed analysis from the 5′ end of ORF7 revealed that a stretch of 34 nucleotides (nucleotides 14653 to 14686) was essential for the expression of the M protein (pABV696; FIG. 1B).

As a control, the mutated ORF7 from pABV696 was replaced by ORF7 of the wild type infectious cDNA clone of LV (pABV730; FIG. 1B). This restored its capability to express the M protein and the other viral structural proteins, demonstrating that the lack of structural protein expression by RNA transcripts from pABV696 had not been caused by unintended mutations elsewhere in the viral genome, possibly introduced during the cloning procedures.

The 34-nucleotide region in ORF7 is essential for RNA replication. To further characterize the role of the 34-nucleotide stretch, we analyzed the effects of its deletion on the synthesis of genomic and subgenomic RNAs of both positive and negative polarity. To obtain a control construct negative for replication, we deleted both ORF7 and the 3′UTR of the LV cDNA clone, yielding pABV668 (FIG. 1A). BHK-21 cells were electroporated in parallel with RNA transcripts from pABV696, pABV437 (positive control), and pABV668 (negative control), respectively. RNA was isolated from these cells 12 hours after transfection. Strand-specific RT-PCR assays were developed to analyze the production of positive and negative strand genomic and subgenomic viral RNA, as outlined in FIGS. 2A1 to A4. Total RNA isolated from cells transfected with transcripts from pABV668 yielded an amplification product only after testing for positive strand genomic RNA (FIG. 2B1). This product was probably derived from the input RNA, because pABV668 lacks the 3′UTR sequences and is therefore unlikely to yield RNA transcripts that are replication competent. When we tested RNA isolated from cells that had been transfected with transcripts from pABV437, RT-PCR products of the expected sizes were obtained for both the genomic positive and negative strand (FIGS. 2B1 and 2B2) and for the sg mRNA7 positive and negative strand (FIGS. 2B3 and 2B4). When we tested RNA from cells transfected with transcripts from pABV696, we obtained similar results as for pABV668 (FIGS. 2B1 to B4). The identity of the PCR products was verified by their size and by restriction enzyme analysis (data not shown).

To further confirm our RT-PCR data, an immunofluorescence assay (IFA) was performed using an antiserum against the nonstructural precursor protein nsp2/3 of PRRSV. Nsp2/3 is translated from genomic RNA, but the level of nsp2/3 produced from non-replicating transcripts is too low to be detected by the antiserum. Therefore, positive staining of nsp2/3 by the antiserum is dependent on RNA replication. In BHK-21 cells transfected with transcripts from pABV696, no expression of nsp2/3 was detected, as was the case with transcripts from our negative control pABV668. In contrast, when using transcripts from our positive control pABV437, we clearly detected the expression of the nsp2/3 precursor protein (data not shown). In conclusion, transcripts from pABV696 were impaired in the synthesis of both positive and negative strand genomic and sg mRNAs. More specifically, the 34-nucleotide stretch in ORF7 appears to be essential for genomic minus-strand RNA synthesis.

The 34-nucleotide Stretch is Highly Conserved in PRRSV Isolates and is Predicted to form a Stem-loop Structure

Sequence comparison of the 34-nucleotide region in ORF7 of LV with that of 133 other PRRSV strains deposited in the GenBank revealed that this sequence is strongly conserved. For most European strains of PRRSV 100% sequence conservation exists, whereas a 97% sequence identity for the complete ORF7 of the European strains was found. The level of homology in the 34-nucleotide stretch between American and European PRRSV strains was slightly lower (about 94%), though still significantly higher than that in the complete ORF7 (about 60%). Secondary structure analysis using the program MFOLD predicted a hairpin within the highly conserved 34-nucleotide region of LV (FIG. 3A). The existence of this hairpin is supported by the sequence data of the other isolates. In four isolates (U64931, L40898, Z82995, and U02095) a neutral variation has occurred, still allowing base pairing. In three isolates (AF121131, AF035409, and L39361) only the bottom base pair is disrupted, which weakens, but not prevents, hairpin formation (data not shown). The nucleotide changes of only one isolate (Ul8750), are not consistent with hairpin formation. We note that in the majority of the isolates, 101 out of 134, the hairpin can even be extended by an additional C-G base pair at the top.

The relatively large size of the loop (10-12 nucleotides) prompted us to look for putative base pairing interactions. We noticed that 7 bases from the loop were complementary to a sequence in the 3′UTR. These complementary nucleotides were predicted to be in the loop of another hairpin, which is also strongly conserved among all PRRSV isolates (FIG. 3B). The reported nucleotide changes do not interfere with hairpin formation: they either fall into single-stranded regions or they preserve base-pairing. Interestingly, the deletion of one C-G base-pair in all American isolates seems to be compensated for by an additional base-pair in the loop-loop interaction due to insertion of a U-residue.

The importance of the 3′UTR hairpin was demonstrated by deletion analysis starting from the PacI site. This site was introduced into the infectious clone directly downstream from the ORF7 stop codon. Deletion of 7 nucleotides downstream of this stop codon had no detrimental effect on replication as indicated by the normal expression of M and N proteins. Extending the deletion 32 nucleotides into the 3′UTR, thereby removing almost the entire hairpin, abolished replication. These results suggested that both the ORF7 and 3′UTR hairpins are important for replication, possibly because they are needed to form the loop-loop interaction as depicted in FIG. 4A.

Kissing Loop Interaction is Required for RNA Replication

To obtain experimental evidence for the proposed kissing, we changed 5 nucleotides from the loop of the ORF7 hairpin into their complement. This was predicted to severely weaken the kissing interaction (FIG. 4B, pABV769). As a result, this mutant failed to replicate as evidenced by the lack of M protein expression in BHK21 cells transfected with its RNA transcripts (FIG. 4B, compare upper left with upper right panel). Similarly, mutating bases in the loop of the 3′UTR hairpin was also detrimental for replication. (FIG. 4B, pABV768, lower left panel). Interestingly, when the ORF7 and 3′UTR hairpin were mutated simultaneously, thus restoring base pairing between the loops, expression of the M protein was again detected (FIG. 4B. lower right panel), indicating that RNA replication was restored. These results clearly demonstrated that the interaction between bases in the loops of ORF7 and the 3′UTR is essential for RNA replication.

The Position of the 34-nucleotide Sequence is Important for its Function

In order to determine whether the relative position of the 34-nucleotide region in the viral genome is important for its function, we relocated it 44 nucleotides upstream. We inserted the 34-nucleotide stretch directly downstream of the stop codon of ORF6 in pABV696, from which the 34-nucleotide region was deleted (pABV697; FIG. 5). Its RNA transcripts, when transfected into BHK-21 cells, showed no detectable expression of the M protein in IPMA. This indicated that the RNA replication and/or transcription could not be restored by relocation of the 34-nucleotide stretch.

Signals regulating the replication of RNA viruses are generally located within the terminal non-coding regions of the genome. Herein we identified and mapped a domain essential for viral replication within a coding region of the porcine arterivirus RNA, the most 3′ORF specifying the viral nucleocapsid protein N. Deletion of this 34-nucleotide domain from genomic RNA completely abolished negative strand RNA synthesis. Theoretical analysis of its sequence predicts it to fold into a stem-loop structure that is highly conserved among porcine arteriviruses. Most interesting, a 7-nucleotide sequence within the loop of this structure appeared to be engaged in a kissing loop interaction with a domain located in the 3′UTR. The latter domain in turn occurs in the loop of a predicted stem-loop structure. Mutation analyses revealed that it is the ability of the two loops to base-pair, not their primary sequences per se, that is functionally relevant. The results suggest that the kissing loop interaction that we observed stabilizes a three-dimensional conformation within the 3′terminal region of the viral genome onto which an RNA polymerase complex can be assembled for the initiation of negative-strand RNA synthesis.

The 34-nucleotide domain critical for PRRSV RNA replication is located in the coding region of the N gene. Thus, if the N protein would have any role in viral RNA replication in addition to its functioning in virus assembly, the effects of deletions in this gene might simply be explained by its debilitating consequences on the protein's functioning. A role of the arterivirus N protein in replication has indeed been suggested on the basis of its co-localization with the polymerase and helicase proteins in the viral replication complex (Pedersen et al., 1999; van der Meer et al., 1999). A similar multifunctional role has also been attributed to the N protein of the related coronaviruses. Here the protein was proposed to be involved in replication (Baric et al., 1988), in transcription (Makino et al., 1986; Shieh et al., 1987), in viral RNA translation as well as in the formation of RNP complexes (Tahara et al., 1994; Tahara et al., 1998). Recently, the domain of the mouse hepatitis virus (MHV) N protein that actually binds its viral RNA has been identified (Nelson et al., 2000). While the significance of these nonstructural roles of the nidovirus N protein remains unclear, these roles are clearly not essential for the replication and transcription of PRRSV RNA. Indeed, our work showed that extensive deletions within the N protein except for the 34-nucleotide stretch did not abolish RNA replication, sg RNA synthesis or viral protein expression. These observations abrogate an essential role for the N protein in any of these processes. This is in accordance with a recent study of EAV, which showed that the structural proteins of EAV are detrimental for genomic RNA replication (Molenkamp et al., 2000b). Our study indicates, however, that the 34-nucleotide sequence present in ORF7 constitutes a cis-acting element.

For a number of positive strand RNA viruses, DI RNAs have been used to map cis-acting sequence elements that participate in replication and transcription. The only arteriviral DI described so far (Molenkamp et al., 2000a) had lost most of its sequences encoding the non-structural proteins, but retained the entire region encoding the structural proteins. Further trimming of this DI genome using a cDNA clone revealed that the 3′terminal 1066 nucleotides were essential either for replication, transcription or packaging. This region included besides the ORF7 gene, the ORF6 gene, as well as the 3′ end of ORF5. Further deletion of the ORF6 and ORF5 genes to establish whether these ORFs are redundant for replication, as demonstrated here for PRRSV, was not performed. So neither the precise sequence requirements nor their function were elucidated. Putative long-distance interactions similar to the LV kissing loop interaction may be located within these 3′ 1066 nucleotides. For coronaviruses, both naturally occurring and artificially constructed DI RNAs have been used to study the genomic regions involved in RNA replication and transcription (Chang et al., 1994; Makino et al., 1988; Mendez et al., 1996; Penzes et al., 1994; van der Most et al., 1991). These analyses showed that the minimal sequence required for DI replication at the 3′ end of the genome is 492 nucleotides for transmissible gastro enteritis virus (Izeta et al., 1999; Mendez et al., 1996) and between 417 and 463 nucleotides for MHV (Kim et al., 1993; Lin and Lai, 1993; Lin et al., 1994; Luytjes et al., 1996; van der Most and Spaan, 1995; Hsue et al., 2000). In both cases this includes the entire 3′UTR and a portion of the upstream ORF, coding for the nucleocapsid protein N. Other studies showed that the minimal sequence requirement for negative-strand RNA synthesis comprises the 3′ most 55 nucleotides of the genome of MHV (Lin et al., 1994), whereas a region encompassing nucleotides 270-305 was required for subgenomic mRNA synthesis. Deletion of this region completely abolished subgenomic mRNA transcription without affecting minus-strand RNA synthesis (Lin et al., 1996). These studies all together indicate that replication and transcription signals of arteri- and coronaviruses are not restricted to the 5′UTR and the 3′UTR, but may also be present in flanking regions encoding structural proteins.

In all PRRSV isolates hairpins are predicted corresponding to the ones that we observed in ORF7 and 3′UTR of LV. This is even so for isolates that show only 60% homology to LV in their ORF7. These hairpins and their kissing interaction are strongly conserved, suggesting a strong selective pressure on sequence and structure. In isolate U87392 (VR2332) the nucleotides of the predicted loops of ORF7 and the 3′UTR that are complementary can be extended to 8 instead of 7 as predicted for LV. This seems to compensate for the deletion of one base pair from the stem of the 3′UTR hairpin. In only 4 of the 133 isolates sequenced today, nucleotide changes in the complementary nucleotides in the loop of the 34-nucleotide stretch were documented. Sequencing of the 3′UTR from these isolates will show whether compensatory mutations are present into their complementary nucleotides of the 3′UTR. No significant homology with the 34-nucleotide stretch of PRRSV was found in the genomes of the other arteriviruses EAV, LDV, and SHFV. However, putative sites of interaction between the 3′UTR and a sequence in the upstream ORF, containing at least 7 complementary bases, were observed (data not shown). Future studies need to identify whether these sequences are used as cis-acting elements for RNA replication of their viral genomes.

The predicted kissing loop interaction between the 34-nucleotide stretch in ORF7 and the 3′UTR is essential for the production of genomic negative strand RNA. The 34-nucleotide stretch might also be essential for the synthesis of negative strand subgenomic RNA, produced from positive strand genomic RNA by a discontinuous transcription mechanism (Baric and Yount, 2000; Sawicki and Sawicki, 1990; Sawicki and Sawicki, 1998; van Marle et al., 1999a). Since ORF7 is located at the 3′ end of the viral genome, the 34-nucleotides might have a role in the formation of complexes for initiation of minus-strand synthesis. Therefore, its position, i.e. the relative distance to the 3′UTR and the adjacent nucleotide sequence, might be important for its structure and therefore for its function. The negative effect of the relocation of the 34-nucleotide stretch on the RNA replication indeed confirms this. At least 10 non-structural viral proteins are involved in RNA replication, and, moreover, host-encoded proteins may take part in the formation of such complex. Protein binding might stabilize the kissing loop interaction, or might prevent or melt the interaction, thereby shutting off minus strand synthesis. An example of such an interaction is the cloverleaf-like secondary RNA structure (trans-activation response element Tar) at the 5′ end of poliovirus. Upon its interaction with the cellular factor Poly(rC) binding protein (PCBP) its viral translation is up-regulated, while its interaction with the viral protein 3CD represses the translation and promotes negative-strand RNA synthesis (Parsley et al., 1997). Proteins are also involved in RNA replication (Hwang and Brinton, 1998; Liu et al., 1997; Yu and Leibowitz, 1995) and transcription (Huang and Lai, 1999) of coronaviruses. However, the ways in which they act are not yet elucidated. Potentially, the interaction with specific proteins might regulate whether the genomic RNA is used for RNA replication or sg mRNA transcription of PRRSV.

Materials and Methods

Cells

BHK-21 cells were grown in BHK-21 medium (Gibco BRL), complemented with 5% FBS, 10% tryptose phosphate broth (Gibco BRL), 20 mM Hepes pH 7.4 (Gibco BRL), 200 mM glutamine, 10 U/ml penicillin, 10 μg/ml streptomycin, 20 μg/ml kanamycin, 5 μg/ml polymixine B, and 0.2 μg/ml fungizone.

Construction of Deletion Mutants of the Full-length Genomic cDNA Clone of LV

Parts of the infectious cDNA clone of LV were deleted using standard cloning techniques. The resulting clones are schematically drawn in FIG. 1A. Nucleotide numbers are based on the LV sequence as deposited by GenBank (Accession Number M96262; (Meulenberg et al., 1998). First, a region comprising ORF2 through the 5′ part of ORF6 was deleted from subclone pABV402, which contains the 3′ two-thirds of the viral genome (Meulenberg et al., 1998). pABV402 was digested with EcoRI and NdeI, treated with Klenow-enzyme, and self-ligated, resulting in subclone pABV593. This subclone was extended to a full-length cDNA clone by insertion of the PmlI-SpeI region of pABV399, which comprises the 5′ one-third of the viral genome (Meulenberg et al., 1998), generating pABV594. Second, ORF7 was deleted from pABV442, a full-length cDNA clone containing a SwaI-site directly downstream of ORF7 (Meulenberg et al., 1998). pABV442 was digested with HpaI and Swal, and self-ligated, generating pABV521. Third, the region comprising ORF2 through 6, except for the TRS of ORF7, was deleted. Plasmid pABV402 was digested with EcoRI and Hpal, treated with Klenow-enzyme, and self-ligated, resulting in subclone pABV663. pABV663 was restored into a full length cDNA clone by insertion of the PmlI-SpeI fragment of pABV399, resulting in pABV664. Fourth, we deleted ORF7 and the 3′UTR from pABV437, a full length cDNA clone containing a PacI-site directly downstream of ORF7 (Meulenberg et al., 1998). pABV437 was digested with HpaI and XbaI, and self-ligation resulted in pABV668.

To introduce smaller deletions into ORF7, PCR-mutagenesis was performed. The generated PCR-fragments were digested with HpaI and PacI, and ligated into pABV437 treated with the same enzymes. The primers used are listed in Table 1, and the resulting clones are depicted in FIG. 1B.

Standard cloning procedures were performed essentially as described (Sambrook, 1989). Transformation conditions were maintained as described (Meulenberg et al., 1998) and sequence analysis was performed to confirm the deletions.

Mutagenesis of the Predicted Loops in ORF7 and the 3′UTR of LV

To introduce mutations in the predicted loops of ORF7 and the 3′UTR, we performed PCR-mutagenesis using the primers detailed in Table 1. The generated 3′UTR fragments were digested with PacI and XbaI and ligated into the similarly digested pABV437. The generated ORF7 fragments were digested with HpaI and PacI, and ligated into the similarly treated pABV437. This resulted in pABV768 containing 5 nucleotide changes in the 3′UTR loop, and in pABV769 containing 5 nucleotide changes in the loop of the 34-nucleotides within ORF7. The double-mutant containing these mutations in both loops was constructed by ligation of the HpaI-PacI fragment from pABV769 into the HpaI-PacI digested pABV768. This resulted in pABV770. The mutations are described in FIG. 4.

Sequence Analysis

The regions of the full-length cDNA clones originating from the PCR products were analyzed by nucleotide sequencing. Sequences were determined with the PRISM Ready Dye Deoxy Terminator cycle sequencing kit and the ABI PRISM 310 Genetic Analyzer (Perkin Elmer).

In Vitro RNA Transcription and Transfection of BHK-21 Cells

The constructed cDNA clones were in vitro transcribed using 1 μg linearized plasmid DNA, and were subsequently treated for 15 minutes with 10 U DNAse at 37° C. BHK-21 cells were transfected with the resulting RNA by electroporation as described (Meulenberg et al., 1998).

Immunoperoxidase Monolayer Assay (IPMA)

Immunostaining of BHK-21 cells was performed essentially as described before (Wensvoort et al., 1986). Monoclonal antibodies (MAbs) against GP3 (122.14), GP4 (122.1), the M protein (126.3) and the N protein (122.17) were used to detect the expression of PRRSV proteins (van Nieuwstadt et al., 1996).

RT-PCR

Twelve hours after transfection, cellular RNA of BHK-21 cells transfected with in vitro transcribed RNA was isolated. Cells were lysed in LET-buffer (100 mM Tris-HCl pH=8.0, 500 mM LiCl, 10 mM EDTA pH=8.0, and 5 mM DTT) containing 20 μg/ml proteinase K for 10 minutes while shaking. The lysates were passed three times through a 25′ Gauge needle using a syringe, and then incubated for 15 minutes at 42° C. The RNA was extracted three times using phenol-chloroform (pH=4.0), once using chloroform, and was then precipitated with isopropanol. The RNA was reverse transcribed as indicated in FIG. 3A. The PCR consisted of 39 cycli, each comprising 30 seconds of denaturation at 94° C., 30 seconds of annealing at 62° C., and 2 minutes of elongation at 72° C. The PCR products were analyzed in 2% agarose gels.

Secondary and Tertiary Structure Analysis

RNA secondary structures were predicted with M. Zuker's Mfold server at www.ibc.wustl.edu/˜zuker/rna/.

References with Example 1

Baric, R. S., Nelson, G. W., Fleming, J. O., Deans, R. J., Keck, J. G., Casteel N. and Stohlman, S. A. (1988) Interactions between coronavirus nucleocapsid protein and viral RNAs: implications for viral transcription. J Virol, 62, 4280-4287.

Baric, R. S. and Yount, B. (2000) Subgenomic negative-strand RNA function during mouse hepatitis virus infection. J Virol, 74, 4039-4046.

Buck, K. W. (1996) Comparison of the replication of positive-stranded RNA viruses of plants and animals. Adv Virus Res, 47, 159-251.

Cavanagh, D. (1997) Nidovirales: A new order comprising Coronaviridae and Arteriviridae. Arch Virol, 142, 629-633.

Chang, R. Y., Hofmann, M. A., Sethna, P. B. and Brian, D. A. (1994) A cis-acting function for the coronavirus leader in defective interfering RNA replication. J Virol, 68, 8223-8231.

de Vries, A. A., Chirnside, E. D., Bredenbeek, P. J., Gravestein, L. A., Horzinek, M. C. and Spaan, W. J. (1990) All subgenomic mRNAs of equine arteritis virus contain a common leader sequence. Nucleic Acids Res, 18, 3241-3247.

Dreher, T. W. (1999) Functions of the 3′-untranslated regions of positive strand RNA viral genomes. Ann Rev Phytopathol, 37, 151-174.

Guan, H., Carpenter, C. D. and Simon, A. E. (2000) Requirement of a 5′-proximal linear sequence on minus strands for plus-strand synthesis of a satellite RNA associated with turnip crinkle virus. Virology, 268, 355-363.

Hsue, B., Hartshorne, T. and Masters, P. S. (2000) Characterization of an essential RNA secondary structure in the 3′ untranslated region of the murine coronavirus genome. J Virol 74, 6911-6921.

Huang, P. Y. and Lai, M. M. C. (1999) Polypyrimidine tract-binding protein binds to the complementary strand of the mouse hepatitis virus 3′ untranslated region, thereby altering RNA conformation. J Virol, 73, 9110-9116.

Hwang, Y. K. and Brinton, M. A. (1998) A 68-nucleotide sequence within the 3′ noncoding region of simian hemorrhagic fever virus negative-strand RNA binds to four MA104 cell proteins. J Virol, 72, 4341-4351.

Izeta, A., Smerdou, C., Alonso, S., Penzes, Z., Mendez, A., Plana Duran, J. and Enjuanes, L. (1999) Replication and packaging of transmissible gastroenteritis coronavirus-derived synthetic minigenomes. J Virol, 73, 1535-1545.

Kim, Y. N., Jeong, Y. S. and Makino, S. (1993) Analysis of cis-acting sequences essential for coronavirus defective interfering RNA replication. Virology, 197, 53-63.

Klovins, J. and van Duin, J. (1999) A long-range pseudoknot in Qbeta RNA is essential for replication. J Mol Biol, 294, 875-884.

Lai, M. M. (1990) Coronavirus: organization, replication and expression of genome. Annu Rev Microbiol, 44, 303-333.

Lai, M. M., Liao, C. L., Lin, Y. J. and Zhang, X. (1994) Coronavirus: how a large RNA viral genome is replicated and transcribed. Infect Agents Dis, 3, 98-105.

Lin, Y. J. and Lai, M. M. (1993) Deletion mapping of a mouse hepatitis virus defective interfering RNA reveals the requirement of an internal and discontiguous sequence for replication. J Virol, 67, 6110-6118.

Lin, Y. J., Liao, C. L. and Lai, M. M. (1994) Identification of the cis-acting signal for minus-strand RNA synthesis of a murine coronavirus: implications for the role of minus-strand RNA in RNA replication and transcription. J Virol, 68, 8131-8140.

Lin, Y. J., Zhang, X., Wu, R. C. and Lai, M. M. (1996) The 3′ untranslated region of coronavirus RNA is required for subgenomic mRNA transcription from a defective interfering RNA. J Virol, 70, 7236-7240.

Liu, Q., Yu, W. and Leibowitz, J. L. (1997) A specific host cellular protein binding element near the 3′ end of mouse hepatitis virus genomic RNA. Virology, 232, 74-85.

Luytjes, W., Gerritsma, H. and Spaan, W. J. (1996) Replication of synthetic defective interfering RNAs derived from coronavirus mouse hepatitis virus-A59. Virology, 216, 174-183.

Makino, S., Shieh, C. K, Soe, L. H., Baker, S. C. and Lai, M. M. (1988) Primary structure and translation of a defective interfering RNA of murine coronavirus. Virology, 166, 550-560.

Makino, S., Stohlman, S. A. and Lai, M. M. (1986) Leader sequences of murine coronavirus mRNAs can be freely reassorted: evidence for the role of free leader RNA in transcription. Proc Natl Acad Sci U S A, 83, 4204-4208.

Melchers, W. J., Bakkers, J. M., Bruins Slot, H. J., Galama, J. M., Agol, V. I. and Pilipenko, E. V. (2000) Cross-talk between orientation-dependent recognition determinants of a complex control RNA element, the enterovirus oriR. Rna, 6, 976-987.

Mendez, A., Smerdou, C., Izeta, A., Gebauer, F. and Enjuanes, L. (1996) Molecular characterization of transmissible gastroenteritis coronavirus defective interfering genomes: packaging and heterogeneity. Virology, 217, 495-507.

Meulenberg, J. J., de Meijer, E. J. and Moormann, R. J. (1993a) Subgenomic RNAs of Lelystad virus contain a conserved leader-body junction sequence. J Gen Virol, 74, 1697-1701.

Meulenberg, J. J., Hulst, M. M., de Meijer, E. J., Moonen, P. L., den Besten, A., de Kluyver, E. P., Wensvoort, G. and Moormann, R. J. (1993b) Lelystad virus, the causative agent of porcine epidemic abortion and respiratory syndrome (PEARS), is related to LDV and EAV. Virology, 192, 62-72.

Meulenberg, J. J., Besten, A. P., De Kluyver, E. P., Moormann, R. J., Schaaper, W. M. and Wensvoort, G. (1995) Characterization of proteins encoded by ORFs 2 to 7 of Lelystad virus. Virology, 206, 155-163.

Meulenberg, J. J., BosDeRuijter, J. N., vandeGraaf, R., Wensvoort, G. and Moormann, R. J. (1998) Infectious transcripts from cloned genome-length cDNA of porcine reproductive and respiratory syndrome virus. J ViroL 72, 380-387.

Molenkamp, R., Rozier, B. C., Greve, S., Spaan, W. J. and Snijder, E. J. (2000a) Isolation and characterization of an arterivirus defective interfering RNA genome. J Virol, 74, 3156-3165.

Molenkamp, R., van Tol, H., Rozier Babette, C. D., van der Meer, Y., Spaan Willy, J. M. and Snijder Eric, J. (2000b) The arterivirus replicase is the only viral protein required for genome replication and subgenomic mRNA transcription. J Gen Virol, 81, 2491-2496.

Nelson, G. W., Stohlman, S. A. and Tahara, S. M. (2000) High affinity interaction between nucleocapsid protein and leader/intergenic sequence of mouse hepatitis virus RNA. J Gen Virol, 1, 181-188.

Olsthoorn, R. C. L., Mertens, S., Brederode, F. T. and Bol, J. F. (1999) A conformational switch at the 3′ end of a plant virus RNA regulates viral replication. Embo J, 18, 4856-4864.

Parsley, T. B., Towner, J. S., Blyn, L. B., Ehrenfeld, E. and Semler, B. L. (1997) Poly (rC) binding protein 2 forms a ternary complex with the 5′-terminal sequences of poliovirus RNA and the viral 3CD proteinase. Rna, 3, 1124-1134.

Pedersen, K. W., van der Meer, Y., Roos, N. and Snijder, E. J. (1999) Open reading frame 1a-encoded subunits of the arterivirus replicase induce endoplasmic reticulum-derived double-membrane vesicles which carry the viral replication complex. J Virol, 73, 2016-2026.

Penzes, Z., Tibbles, K., Shaw, K., Britton, P., Brown, T. D. and Cavanagh, D. (1994) Characterization of a replicating and packaged defective RNA of avian coronavirus infectious bronchitis virus. Virology, 203, 286-293.

Sagripanti, J. L., Zandomeni, R. O. and Weinmann, R. (1986) The cap structure of simian hemorrhagic fever virion RNA. Virology, 151, 146-150.

Sambrook, J., Fritsch, E. F., Maniatis, T. (1989) Molecular cloning: a laboratory manual, 2nd ed. Cold Spring Harbor Laboratory, Cold Spring Harbor, N.Y.

Sawicki S. G. and Sawicki, D. L. (1990) Coronavirus transcription: subgenomic mouse hepatitis virus replicative intermediates function in RNA synthesis. J Virol, 64, 1050-1056.

Sawicki, S. G. and Sawicki, D. L. (1998) A new model for transcription. In L. Enjuanes, S. G. S., and W. J. M. Spaan (ed.), Coronaviruses and Arteriviruses. Plenum Press, New York, N.Y., pp. 215-219.

Shieh, C. K, Soe, L. H., Makino, S., Chang, M. F., Stohlman, S. A. and Lai, M. M. (1987) The 5′-end sequence of the murine coronavirus genome: implications for multiple fusion sites in leader-primed transcription. Virology, 156, 321-330.

Snijder, E. J. and Meulenberg, J. J. (1998) The molecular biology of arteriviruses. J Gen Virol, 79, 961-979.

Snijder, E. J., van Tol, H., Pedersen, K. W., Raamsman, M. J. and de Vries, A. A. (1999) Identification of a novel structural protein of arteriviruses. J Virol, 73, 6335-6345.

Snijder, E. J., Wassenaar, A. L. and Spaan, W. J. (1994) Proteolytic processing of the replicase ORF1a protein of equine arteritis virus. J Virol, 68, 5755-5764.

Tahara, S. M., Dietlin, T. A., Bergmann, C. C., Nelson, G. W., Kyuwa, S., Anthony, R. P. and Stohlman, S. A. (1994) Coronavirus translational regulation: leader affects mRNA efficiency. Virology, 202, 621-630.

Tahara, S. M., Dietlin, T. A., Nelson, G. W., Stohlman, S. A. and Manno, D. J. (1998) Mouse hepatitis virus nucleocapsid protein as a translational effector of viral mRNAs. Adv Exp Med Biol, 440, 313-318.

van der Meer, Y., Snijder, E. J., Dobbe, J. C., Schleich, S., Denison, M. R., Spaan, W. J. and Locker, J. K. (1999) Localization of mouse hepatitis virus nonstructural proteins and RNA synthesis indicates a role for late endosomes in viral replication. J Virol, 73, 7641-7657.

van der Most, R. G., Bredenbeek, P. J. and Spaan, W. J. (1991) A domain at the 3′ end of the polymerase gene is essential for encapsidation of coronavirus defective interfering RNAs. J Virol, 65, 3219-3226.

van der Most, R. G. and Spaan, W. J. M. (1995) Coronavirus replication, transcription, and RNA recombination. In Siddell, S. G. (ed.), The Coronaviridae. Plenum Press, New York, N.Y., pp. 11-31.

van Dinten, L. C., Rensen, S., Gorbalenya, A. E. and Snijder, E. J. (1999) Proteolytic processing of the open reading frame 1b-encoded part of arterivirus replicase is mediated by nsp4 serine protease and Is essential for virus replication. J ViroL 73, 2027-2037.

van Dinten, L. C., Wassenaar, A. L., Gorbalenya, A. E., Spaan, W. J. and Snijder, E. J. (1996) Processing of the equine arteritis virus replicase ORF1b protein: identification of cleavage products containing the putative viral polymerase and helicase domains. J Virol, 70, 6625-6633.

van Marle, G., Dobbe, J. C., Gultyaev, A. P., Luytjes, W., Spaan, W. J. M. and Snijder, E. J. (1999a) Arterivirus discontinuous mRNA transcription is guided by base pairing between sense and antisense transcription-regulating sequences. Proc Natl Acad Sci USA, 96, 12056-12061.

van Marle, G., Luytjes, W., van der Most, R. G., van der Straaten, T. and Spaan, W. J. (1995) Regulation of coronavirus mRNA transcription. J Virol, 69, 7851-7856.

van Marle, G., van Dinten, L. C., Spaan, W. J., Luytjes, W. and Snijder, E. J. (1999b) Characterization of an equine arteritis virus replicase mutant defective in subgenomic mRNA synthesis. J Virol, 73, 5274-5281.

van Nieuwstadt, A. P., Meulenberg, J. J., van Essen Zanbergen, A., Petersen den Besten, A., Bende, R. J., Moormann, R. J. and Wensvoort, G. (1996) Proteins encoded by open reading frames 3 and 4 of the genome of Lelystad virus (Arteriviridae) are structural proteins of the virion. J Virol, 70, 4767-4772.

Wassenaar, A. L., Spaan, W. J., Gorbalenya, A. E. and Snijder, E. J. (1997) Alternative proteolytic processing of the arterivirus replicase ORF1a polyprotein: evidence that NSP2 acts as a cofactor for the NSP4 serine protease. J Virol, 71, 9313-9322.

Wensvoort, G., Terpstra, C., Boonstra, J., Bloemraad, M. and Van Zaane, D. (1986) Production of monoclonal antibodies against swine fever virus and their use in laboratory diagnosis. Vet Microbiol, 12, 101-108.

Wensvoort, G., Terpstra, C., Pol, J. M., ter Laak, E. A., Bloemraad, M., de Kluyver, E. P., Kragten, C., van Buiten, L., den Besten, A., Wagenaar, F. and et al. (1991) Mystery swine disease in The Netherlands: the isolation of Lelystad virus. Vet Q, 13, 121-130.

Williams, G. D., Chang, R. Y. and Brian, D. A. (1995) Evidence for a pseudoknot in the 3′ untranslated region of the bovine coronavirus genome. Adv Exp Med Biol, 380, 511-514.

Yu, W. and Leibowitz, J. L. (1995) Specific binding of host cellular proteins to multiple sites within the 3′ end of mouse hepatitis virus genomic RNA J Virol, 69, 2016-2023.

### EXAMPLE 2

In example 2, we focus on the 3′ end of the PRRSV genome since this region does not contain sequences overlapping with other ORFs. Until now, the deletions that were introduced in the N-terminal and middle part of the coding region of the N protein did not result in viable virus (Verheije, M. H., unpublished results).

Methods

Cells and viruses. BHK-21 cells were grown in BHK-21 medium (Gibco BRL) complemented with 5% FBS, 10% tryptose phosphate broth (Gibco BRL), 20 mM Hepes pH 7.4 (Gibco BRL), 200 mM glutamine, 10 U/ml penicillin, 10 μg/ml streptomycin, 20 μg/ml kanamycin, 5 μg/ml polymixine B, and 0.2 μg/ml fungizone. Porcine. alveolar lung macrophages (PAMs) were maintained in MCA-RPMI-1640 medium containing 10% FBS, 100 μg/ml kanamycin, 50 U/ml penicillin, 50 μg/ml streptomycin, 25 μg/ml polymixine B, and 1 μg/ml fungizone. Serial passage of the recombinant PRRS viruses was performed by inoculation of 500 μl of the culture. supernatant of transfected BHK-21 cells onto 1×107 PAMs. The inoculum was removed after 1 hour and 5 ml of fresh medium was added. The culture supernatant containing the produced virus was harvested when the first signs of cytopathogenic effect (cpe) were observed, generally around 48 hours after infection. The virus was further passaged by repeatedly inoculating 500 μl of the harvested culture medium of the previous passage onto 1×107 PAMs and again harvesting the culture supernatant after 48 hours. Virus titres (expressed as 50% tissue culture infective doses [TCID50] per ml) were determined on PAMs by end point dilution (Wensvoort et al., 1986).

Construction of full-length genomic cDNA clones of LV. PCR-mutagenesis was used to introduce sequences into the PacI-mutant of the genome-length cDNA clone of LV (pABV437) (Meulenberg et al., 1998a). The primers used for PCR-mutagenesis are listed in Table 1. PCR-fragments generated to introduce deletions into ORF7 were digested with HpaI and PacI, and ligated into these sites of pABV437. PCR-fragments generated to introduce deletions into the 3′UTR were digested with PacI and Xbal, and ligated into these sites of pABV437. Standard cloning procedures were performed essentially as described (Sambrook, 1989). Transformation conditions were maintained as described (Meulenberg et al., 1998a). Sequence analysis was performed to confirm the introduced mutations. The constructs are schematically drawn in FIG. 7

Sequence analysis. The regions of the full-length cDNA clones originating from the PCR products were analysed by nucleotide sequencing. Sequences were determined with the PRISM Ready Dye Deoxy Terminator cycle sequencing kit and the ABI PRISM 310 Genetic Analyser (Perkin Elmer).

In vitro transcription and transfection of BHK-21 cells. The full-length genomic cDNA clones were in vitro transcribed and the resulting RNA was transfected into BHK-21 cells either using Lipofectin (Gibco BRL) or by electroporation (Meulenberg et al., 1998a).

Immunoperoxidase monolayer assay (IPMA). Immunostaining of BHK-21 cells and PAMs was performed by the methods described (Wensvoort et al., 1986). Monoclonal antibodies (MAbs) against GP3 (122.14), GP4 (122.1), the M protein (126.3; (van Nieuwstadt et al., 1996), and against the different antigenic domains of the N protein (138.22 (domain A), 126.9 (domain B), 126.15 (domain C), and 122.17 (domain D; (Meulenberg et al., 1998b) were used to detect the expression of PRRSV proteins.

Infection of PAMs. To rescue infectious virus, the culture supernatant of BHK-21 cells was harvested 24 hours after transfection and used to inoculate PAMs. After 1 hour, the inoculum was removed and fresh culture medium was added. Approximately 15 hours after infection the culture supernatant was harvested and PAMs were washed with PBS, dried and stored at −20° C. until IPMA was performed.

Genetic analysis of genomic RNA of recombinant viruses. To analyse the viral RNA in the culture supernatant of PAMs and in the fractions of the sucrose gradient, 200 μl of the culture supernatant or of the fraction was diluted with an equal volume of proteinase K buffer (100 mM Tris-HCl [pH 7.2], 25 mM EDTA, 300 mM NaCl, 2% [wt/vol] sodium dodecyl sulfate), and 0.08 mg proteinase K was added. After incubation for 30 minutes at 37° C., the RNA was extrac ted with phenol-chloroform and precipitated with ethanol. The RNA was reverse transcribed with primer LV76, and PCR was performed using primers 119R218R and LV20 flanking the region of the viral genome containing the deletions. The amplified fragments were analysed in 2% agarose gels, the PCR fragments were excised from the gel and purified with SpinX columns (Costar). Sequence analysis of the fragments was performed using the antisense primer of the PCR.

Radioimmunoprecipitation (RIP). Metabolic labelling and immunoprecipitation of proteins expressed in PAMs was performed essentially as described (Meulenberg & Petersen den Besten, 1996). MAb 122.17 was used to immunoprecipitate the N protein. PAMs were infected with passage 5 of the viruses at a multiplicity of infection of 1, and were labelled for 4 hours with Tran[35-S]-label (Amersham) at 15 hours post infection. Samples were analysed by sodium dodecyl sulfate polyacrylamide gel electrophoresis (SDS-PAGE) using a 14% acrylamide gel.

Virus concentration and purification. To analyse the production of (noninfectious) virus particles, BHK-21 cells were electroporated with RNA transcripts from pABV747 and pABV437, and 15 hours after transfection the cells were metabolically labelled with 75 μl (10.5 mCi/ml) Tran[35-S]-label (Amersham) for 24 hours (Meulenberg & Petersen den Besten, 1996). The particles in the supernatant were concentrated by centrifuging the supernatant through a 0.5 M sucrose cushion at 26,000 rpm for 5 hours at 4° C. (Meulenberg & Petersen den Besten, 1996). The pellet was resuspended in TNE buffer (0.01 M Tris-HCl, pH7.2; 0.1 M NaCl; and 1 mM EDTA, pH 8.0) and layered onto a 20-50% sucrose gradient (van Berlo et al., 1982). The sucrose gradient was centrifuged at 32,000 rpm for 19 hours at 4° C. Fractions of 0.5 ml were collected from bottom to top and 5 μl of the fractions were analysed by SDS-PAGE using a 14% acrylamide gel.

Results

Sequence Comparison of the N Protein and of the 3′UTR of PRRSV Strains LV and VR2332

Since PRRSV is an RNA virus with a very concise genome, most of its genetic information is expected to be essential. Therefore, genomic cDNA clones containing deletions—especially in the conserved regions—generally do not produce infectious transcripts (Verheije, M. H., unpublished results). In order to identify regions of heterogeneity, where deletions might be tolerated, sequence comparisons were performed. The ORF7 gene at the 3′ end of the LV genome was selected because this ORF does not overlap with other ORFs. Amino acid alignments of the N protein sequence encoded by ORF7 of the prototype European strain (Lelystad virus, LV) and the prototype North-American strain (VR232) showed 60% overall homology (recently reviewed by (Dea et al., 2000)). At the C-terminus of the N protein, the amino acid sequence is highly conserved up till residue 119 of LV. Downstream of this conserved region, a short stretch without amino acid conservation occurs. In addition, the N protein of LV is 4 amino acids longer than that of VR2332 (FIG. 6A). It was therefore anticipated that deletions in the heterogeneous C-terminus of the N protein of LV might be tolerated, and this region was selected as a target to introduce deletions.

Further nucleotide sequence comparison of the 3′UTR downstream of the ORF7 gene also revealed interesting differences (Allende et al., 1999). The 11 most 5′ nucleotides of the 3′UTR of LV show no homology with the first nucleotides of VR2332 (FIG. 6B). Directly downstream of these nucleotides, a stretch of 38 nucleotides is present in VR2332, which has no counterpart in LV. In contrast, high sequence conservation was observed further downstream. In view of this heterogeneity, the region directly downstream of the stop codon of ORF7 was also selected as a target site for deletion studies.

LV Accepts C-terminal Truncations of up to 6 Amino Acids of the N Protein.

cDNA clones with deletions in the sequence coding for the two (pABV639), four (pABV694), and nine (pABV695) C-terminal amino acids of the N protein were constructed by PCR-mutagenesis and cloning of the PCR-fragments into the infectious cDNA clone of LV containing a PacI-site at the stop codon of ORF7 (Meulenberg et al., 1998a) (FIG. 7). The RNA transcripts of these constructs were transfected into BHK-21 cells and tested for their ability to replicate by analysing the expression of the structural proteins in IPMA (FIG. 7). All transcripts expressed the viral proteins GP3, GP4, and M. To analyse the expression of the N protein, and in particular its antigenic domains (Meulenberg et al., 1998b), we used the MAbs 138.22 against the antigenic domain A, 126.9 against domain B, 126.15 against domain C, and 122.17 against domain D of the protein in IPMA. For all constructs, we found that the transfected cells could be stained with each of the MAbs. These results indicated that LV genomes containing deletions at the C-terminus of the N protein still replicated and that the structural proteins were properly translated. In addition, these deletions did not disturb the N protein's antigenic domains.

To investigate whether the LV mutants with a C-terminally truncated N protein produced infectious virus, we inoculated PAMs with the culture supernatants of the transfected BHK-21 cells, as PRRSV cannot infect BHK-21 cells. Twenty-four hours later, the cells were fixed and stained with PRRSV-specific MAbs. PAMs inoculated with the supernatant of BHK-21 cells transfected with transcripts of pABV437, pABV639, and pABV694 stained positive. In contrast, no staining of PAMs was observed after inoculation with supernatant from BHK-21 cells transfected with RNA transcripts from pABV695. In conclusion, LV mutants producing an N protein with a C-terminal deletion of up to 4 produce infectious virus, whereas mutants producing an N protein with a C-terminal deletion of 9 amino acids do not produce infectious virus at all.

To further define the acceptable limits of truncation of the N protein, we made stepwise deletions in the region coding for the 5 to 8 most C-terminal amino acids. The fragments generated by PCR-mutagenesis were again introduced into pABV437, resulting in pABV745, 746, 747, and 748 coding for N proteins lacking 5, 6, 7, and 8 C-terminal amino acids, respectively (FIG. 7). Transfection of their RNA transcripts into BHK-21 cells resulted in the expression of the structural proteins for all constructs, as detected by IPMA. After infection of PAMs with the culture supernatant of the transfected BHK-21 cells, we only detected expression of the structural proteins for vABV745 and vABV746. For mutants lacking the region coding for the C-terminal 7 amino acids or more, no staining was observed in IPMA.

These results indicate that the maximum region that can be deleted at the 3′ end of ORF7 without abolishing the production of infectious virus comprises 18 nucleotides encoding the 6 C-terminal residues of the N protein. The virus produced by this deletion mutant (vABV746) was found to express the set of N protein epitopes as demonstrated using our panel of monoclonal antibodies (data not shown).

Deletion of 7 but not of 32 nucleotides at the 5′ end of the 3′UTR of LV are tolerated In view of the observed nucleotide sequence variation in the 3′UTR of the PRRSV genome downstream of ORF7 (FIG. 6B), we also investigated how deletion of these nucleotides would affect the infection process. Deletions were again introduced by PCR-mutagenesis and the PCR-fragments were introduced into pABV437, directly behind the PacI-site at the stop codon of ORF7. The first 4 nucleotides of the 3′UTR were left intact, as they are part of this PacI-site. This resulted in the plasmids pABV693, which has a deletion of 7 nucleotides, and pABV729, in which a deletion of 32 nucleotides occurs at the 5′ end of the 3′UTR. BHK-21 cells transfected with transcripts of pABV693 expressed the structural proteins. However, BHK-21 cells transfected with transcripts of pABV729 did not express these structural proteins to levels detectable by IPMA, suggesting that RNA replication and/or transcription did not occur. Subsequent infection of PAMs with the culture supernatant of the BHK-21 cells that had been transfected with pABV693 showed expression of the structural proteins in IPMA 24 hours after infection. These results demonstrated that at least 7 nucleotides at the 5′ end of the 3′UTR are dispensable for the virus to remain infectious.

Analysis of the stability and growth characteristics of vABV746 and vABV693 in vitro In order to investigate whether the deletions in the viruses generated from pABV746 and pABV693 were stably maintained in vitro, these viruses were serially passaged on PAMs. After 5 passages, viral RNA was isolated from the culture supernatant and studied by genetic analysis. The RNA was reverse transcribed and the region flanking the introduced deletions was amplified by PCR. Sequence analysis of the fragment showed that in either case the introduced deletion was still present (data not shown) and that no additional mutations had been introduced in the flanking regions. These results indicated that the deletions had been stably maintained during in vitro passaging on PAMs.

The growth characteristics of the viruses vABV746 and vABV693 were investigated by determining their growth curves and comparing them with that of wild type vABV437. PAMs were infected with viruses from passage 5 at a multiplicity of infection of 0.05, and samples were taken from the culture media at various time points. Virus titres were determined by end point dilution on macrophages. As is clear from FIG. 8, no significant differences in growth rates could be observed between recombinant viruses and wild type virus.

Analysis of the Truncated N Protein of vABV746

To confirm the effect of the deletion at the protein level, the size of the N protein expressed by the recombinant virus vABV746 was analysed by immunoprecipitation. PAMs were infected, metabolically labelled with 35S-amino acids, and cell lysates were prepared. The N protein in the lysates was precipitated with MAb 122.17, which is directed against the D-domain of the protein and analysed by SDS-PAGE. As expected, an N protein of wild type size (15 kDa) was immunoprecipitated from lysates of cells transfected with vABV437 (FIG. 9), whereas a protein with an estimated size of 14 kDa was immunoprecipitated from vABV746 infected cell lysates (FIG. 9). The smaller N protein expressed by vABV746 is consistent with the 6-residue truncation as compared to wild type vABV437.

Analysis of Particle Assembly after N Protein Truncation

The abrupt transition in viability upon deletion of more than 6 C-terminal amino acids may have various reasons. We analysed whether the life cycle of the virus was disturbed at the stage of virus assembly. Therefore, BHK-21 cells were transfected with RNA transcripts from pABV747 and proteins were labelled for 24 hours starting at 15 hours after transfection. Viral particles released into the culture supernatant were concentrated by centrifugation through a sucrose cushion, further purified by equilibrium centrifugation in a sucrose gradient, and fractions of this gradient were analysed by electrophoresis of the proteins in SDS-PAG. Structural proteins of the appropriate size were detected for the positive control pABV437 in fractions 5, 6, and 7. These structural proteins were, however, not detected in any of the fractions of pABV747 (FIG. 10A). To confirm the absence of packaged RNA in these fractions, we isolated the viral RNA from each fraction and performed RT-PCR. In none of the fractions was a PRRSV-specific PCR-fragment detected, in contrast to the gradient run with pABV437-derived material (FIG. 10B). In each of these fractions, two PCR-fragments were observed, in contrast to the lane in which we used CDNA of pABV437 as a template. The nature of the second band derived from the fractions could, however, not be identified. The lack of virus particles or virus-like particles produced by transcripts of pABV747 suggests that virus assembly is disturbed in this mutant.

With the purpose to obtain live attenuated PRRSV vaccines, we describe the construction and analysis of several viral deletion mutants. In view of the genetic variability at the 3′ end of the PRRSV genome, we have tested the effect of deletions in this variable region. We report that constructs lacking the coding sequence for up to 6 C-terminal amino acids of the LV N protein still yielded infectious virus after transfection of their transcripts into BHK-21 cells. In contrast, further deletions were fully detrimental: the removal of just one additional residue abolished the production of viable virus completely. Furthermore, also directly downstream of the stop codon of ORF7 were deletions tolerated. At least 7 nucleotides in this region were dispensable for virus production; removal of 32 nucleotides was, however, fatal. Both the virus with a 6-amino acid truncation of the N protein and the virus with the 7 nucleotide deletion in the 3′UTR had in vitro growth characteristics and antigenic profiles similar to that of wild type virus. Moreover, these viruses were both genetically stable.

The dramatic effect of truncation at the 7th residue of the LV N protein was quite surprising, and was not predicted by the sequence. The C-terminal 9 residues sequence of the LV N protein is very different from that of the VR2332 isolate except for its high content of hydroxyl amino acids. In the LV and VR2332 N protein 6 out of 10 residues and 3 out of 6 residues at the very C-terminus, respectively, are serines or threonines. The function of this domain and of these particular residues is unknown. Also two other arteriviruses, LDV and SHFV, contain hydroxyl amino acids at the extreme C-terminus of their N protein, namely 3 out of 10 and 4 out of 10 amino acids, respectively. In contrast, hydroxyl amino acids are fully lacking in the last 10 amino acids of the EAV N protein. While coronavirus N proteins generally do have a relatively high serine content (7-11%) (Masters & Sturman, 1990), the proportion of serines and threonines at their carboxy terminus is quite insignificant; in these viruses this region is markedly acidic. Obviously, these variable. characteristics do not allow predictions for the role of the C-terminus of the N protein in the viral life cycle. The truncated N protein had the same antigenic profile as the wild type N protein, since it reacted with all MAbs directed against antigenic domains of the N protein. This is consistent with observations by Meulenberg et al. (1998b), who identified that domain D, the most C-terminal domain of N, is a conformation dependent or discontinuous epitope that involves amino acids 51-67 and 80-90.

Viral particle production appeared to be blocked after truncation of the LV N protein by 7 amino acids. This strongly indicates a defect at the level of virus assembly. For a Canadian PRRSV isolate, it has been demonstrated that non-covalent interactions between the C-terminal regions of N proteins are critical for formation of the isometric capsid protein (Wootton & Yoo, 1999). In a system expressing only the N protein, they showed that the last 11 amino acids were involved in these interactions. This might indicate that the C-terminus of PRRSV.is essential for nucleocapsid formation. Our study supports this idea. Other effects of C-terminal truncation of the N protein can, however, not be excluded as the N protein has been implicated in various other processes, such as interaction with the viral RNA ((Dea et al., 2000), for MHV (Cologna & Hogue, 1998, Molenkamp & Spaan, 1997), and interaction with other viral proteins (for MHV (Narayanan et al., 2000). Since for Mouse Hepatitis Virus (MHV), the best studied coronavirus, it has been described that a 29-amino acid deletion in the putative spacer region preceding the C-terminal domain of the N protein resulted in temperature sensitive and thermolabile viruses (Peng et al., 1995), we investigated whether our deletion mutants had similar characteristics, which they appeared not to have. Moreover, infectious virus was still not produced from the deletion mutants expressing truncated N proteins lacking 7 amino acids or more after lowering the incubation temperature to 30° C. In an earlier study, we demonstrated that extension of the C-terminus of the N protein by a 9 amino acid sequence of the influenza virus HA protein (Groot Bramel-Verheije et al., 2000) significantly impaired viral growth. We could, however, not establish whether this was caused by the disturbance of virus assembly or of disassembly. Again, these observations are consistent with the C-terminal region of the LV N protein being involved in N-N interactions essential for the production of nucleocapsids during virus assembly.

RNA viruses have at their termini non-coding sequences that play essential roles in RNA replication and sg mRNA transcription. Mutations in these domains are likely to affect the virus life cycle. Consistently, when we introduced deletions in the 5′ terminal region of the LV 3′UTR we found out that removal of a small 7-nucleotides variable sequence was accepted, while removal of a somewhat larger, 32-nucleotide stretch was not. From the inability of the RNA transcripts to express the M and N protein, we conclude that the defect likely resides in an effect on RNA replication or sg mRNA transcription. This suggests that this region of the 3′UTR probably contains an essential RNA signal. Our results are in accordance with studies on coronaviruses that showed that the 5′-terminus of the 3′UTR is essential in the initial processes of the viral life cycle (Hsue et al., 2000). No host or viral proteins were found to specifically bind this region of the viral RNA. However, the exact function of this region still remains to be elucidated.

Herein we aimed at generating viable PRRSV mutants with maximal deletions at the target site. The viruses obtained were characterised in vitro, and fulfilled the most important requirements, good growth and genetic stability. Because their in vitro growth characteristics on PAMs were identical to those of wild type virus, virus production for in vivo studies can easily be accomplished. The growth characteristics in vitro do not necessarily correlate with or predict the behaviour of the virus in vivo. Thus, many currently used vaccines are attenuated in vivo, but show no differences in in vitro propagation (Yang et al., 1998). Therefore, only animal experiments will tell how these viruses behave in vivo, whether they are sufficiently attenuated and whether they induce immune responses that will protect against infection with virulent PRRSV.

References with Example 2

Allende, R., Lewis, T. L., Lu, Z., Rock, D. L., Kutish, G. F., Ali, Ali, Doster, A. R. & Osorio, F. A. (1999). North American and European porcine reproductive and respiratory syndrome viruses differ in non-structural protein coding regions. Journal of General Virology 80, 307-315.

Cavanagh, D. (1997). Nidovirales: A new order comprising Coronaviridae and Arteriviridae. Archives of Virology 142, 629-633.

Cologna, R. & Hogue, B. G. (1998). Coronavirus nucleocapsid protein. RNA interactions. Advances in Experimental Medicine and Biology 440, 355-359.

de Vries, A. A., Chirnside, E. D., Bredenbeek, P. J., Gravestein, L. A., Horzinek, M. C. & Spaan, W. J. (1990). All subgenomic mRNAs of equine arteritis virus contain a common leader sequence. Nucleic Acids Research 18, 3241-3247.

Dea, S., Gagnon, C. A., Mardassi, H., Pirzadeh, B. & Rogan, D. (2000). Current knowledge on the structural proteins of porcine reproductive and respiratory syndrome (PRRS) virus: comparison of the North American and European isolates. Archives of Virology 145, 659-688.

Groot Bramel-Verheije, M. H., Rottier, P. J. M. & Meulenberg, J. J. M. (2000). Expression of a foreign epitope by porcine reproductive and respiratory syndrome virus. Virology 278, 380-389.

Hsue, B., Hartshorne, T. & Masters, P. S. (2000). Characterization of an essential RNA secondary structure in the 3′ untranslated region of the murine coronavirus genome. Journal of Virology 74, 6911-6921.

Masters, P. S. & Sturman, L. S. (1990). Functions of the coronavirus nucleocapsid protein. In Coronaviruses and Their Diseases, pp. 235-238. Edited by D. Cavanagh & T. D. K. Brown. New York: Plenum Press.

Meulenberg, J. J., Bos de Ruijter, J. N., van de Graaf, R., Wensvoort, G. & Moormann, R. J. (1998a). Infectious transcripts from cloned genome-length cDNA of porcine reproductive and respiratory syndrome virus. Journal of Virology 72, 380-387. Meulenberg, J. J., de Meijer, E. J. & Moormann, R. J. (1993a). Subgenomic RNAs of Lelystad virus contain a conserved leader-body junction sequence. Journal of General Virology 74, 1697-1701.

Meulenberg, J. J., Hulst, M. M., de Meijer, E. J., Moonen, P. L., den Besten, A., de Kluyver, E. P., Wensvoort, G. & Moormann, R. J. (1993b). Lelystad virus, the causative agent of porcine epidemic abortion and respiratory syndrome (PEARS), is related to LDV and EAV. Virology 192, 62-72.

Meulenberg, J. J. & Petersen den Besten, A. (1996). Identification and characterization of a sixth structural protein of Lelystad virus: the glycoprotein GP2 encoded by ORF2 is incorporated in virus particles. Virology 225, 44-51.

Meulenberg, J. J., van Nieuwstadt, A. P., van Essen Zandbergen, A, Bos de Ruijter, J. N., Langeveld, J. P. & Meloen, R. H. (1998b). Localization and fine mapping of antigenic sites on the nucleocapsid protein N of porcine reproductive and respiratory syndrome virus with monoclonal antibodies. Virology 252, 106-114.

Meulenberg, J. J. M., Besten, A P. D., De Kluyver, E. P., Moormann, R. J. M., Schaaper, W. M. M. & Wensvoort, G. (1995). Characterization of proteins encoded by ORFs 2 to 7 of Lelystad virus. Virology 206, 165-163.

Molenkamp, R. & Spaan, W. J. (1997). Identification of a specific interaction between the coronavirus mouse hepatitis virus A59 nucleocapsid protein and packaging signal. Virology 239, 78-86.

Narayanan, K, Maeda, A., Maeda, J. & Makino, S. (2000). Characterization of the coronavirus M protein and nucleocapsid interaction in infected cells. Journal of Virology 74, 8127-8134.

Peng, D., Koetzner, C. A & Masters, P. S. (1995). Analysis of second-site revertants of a murine coronavirus nucleocapsid protein deletion mutant and construction of nucleocapsid protein mutants by targeted RNA recombination. Journal of Virology 69, 3449-3457.

Sambrook, J., Fritsch, E. F., Maniatis, T. (1989). Molecular cloning: a laboratory manual, 2nd ed.: Cold Spring Harbor Laboratory, Cold Spring Harbor, N.Y. Snijder, E. J., van Tol, H., Pedersen, K. W., Raamsman, M. J. & de Vries, A. A. (1999). Identification of a novel structural protein of arteriviruses. Journal of Virology 73, 6335-6345.

van Berlo, M. F., Horzinek, M. C. & van der Zeijst, B. A. (1982). Equine arteritis virus-infected cells contain six polyadenylated virus-specific RNAs. Virology 118, 345-352.

van Nieuwstadt, A. P., Meulenberg, J. J., van Essen Zanbergen, A., Petersen den Besten, A., Bende, R. J., Moormann, R. J. & Wensvoort, G. (1996). Proteins encoded by open reading frames 3 and 4 of the genome of Lelystad virus (Arteriviridae) are structural proteins of the virion. Journal of Virology 70, 4767-4772.

Wensvoort, G., Terpstra, C., Boonstra, J., Bloemraad, M. & Van Zaane, D. (1986). Production of monoclonal antibodies against swine fever virus and their use in laboratory diagnosis. Veterinary Microbiology 12, 101-108.

Wootton, S. K. & Yoo, D. (1999). Structure-function of the ORF7 protein of porcine reproductive and respiratory syndrome virus in the viral capsid assembly. In Proceedings of the International Symposium on PRRS and Aujeszky's Disease, pp. 37-38. Ploufragan, France.

Yang, S. X., Kwang, J. & Laegreid, W. (1998). Comparative sequence analysis of open reading frames 2 to 7 of the modified live vaccine virus and other North American isolates of the porcine reproductive and respiratory syndrome virus. Archives of Virology 143, 601-612.

### Vaccination Examples

Intranasal Inoculation of Wild-type PRRSV (EU en US-type) after Vaccination of 8-week Old Pigs with Specified PRRSV-mutants; Virus Kinetics and Antibody Response

Introduction

The Porcine Reproductive and Respiratory Syndrome Virus (PRRSV) causes abortion and poor litter quality in third trimester pregnant sows. Moreover, it may cause respiratory disease in young pigs. Infection of late term pregnant sows (80-95 days) with PRRSV can cause profound reproductive failure, especially due to a high level of mortality among the off-spring of these sows at birth and during the first week after birth. PRRSV is a ubiquitous pathogen. Two distinct antigenic types can be distinguished, i.e. the European and the American type. Clinical effects after a PRRSV infection depend on the type of strain involved. Vaccination of pigs with a PRRS vaccine influences the way a PRRSV-challenge works out on an animal and a farm level. The level and duration of viraemia, and shedding of the field-virus is reduced by this vaccination.

For the development of a second generation PRRS vaccine, new candidates are to be tested. Therefore, 8-week old pigs were vaccinated with a number of specified PRRSV-mutants (recombinant viruses), after which a PRRSV-challenge was given. Kinetics of this virus exposure is scored in terms of level and duration of viremia and booster responses, both in a homologous and heterologous set-up.

Aims of the Study

The determination of the immunological efficacy and safety of defined PRRSV-mutants used as a vaccine in a vaccination-(homologous and heterologous) challenge model. Along with this, mutant immunogenicity was tested.

Study Design

Four PRRSV mutants were tested which all full-filled the following criteria:

genetic stability after 5 passages in-vitro (cell cultures)

genetic stability after 3 weeks of exposure to animals

immunogenicity (as determined by IDEXX elisa)

The following mutants were tested:

vABV707: LDV-PRRS chimeric virus (ectodomain of M exchange)

vABV741: aa9 deletion of the M-protein of PRRSV

vABV746: 18 nucleotide deletions at the C-terminal part of ORF7

vABV688: mutations at position 88-95 of ORF2

As a Positive Control, the Following Virus was Used:

vABV437: wild-type recombinant of Lelystad virus

Each Mutant was Tested in Two Groups each Consisting of 5 SPF-pigs of 8 Weeks Old.

All groups were completely segregated without any contact with each other. Two naive sentinel pigs (so, one per each mutant-group) were united with these vaccinated pigs 24 hours after vaccination and removed and killed 28 days thereafter.

In the 2 groups (per mutant) each consisting of 5 vaccinates, two animals were challenged with wild-type virus (i.e. Lelystad virus (LV-tH) as a representative of an European strain of PRRSV or SDSU#73 as a representative of an American (US) strain of PRRSV), at day 28 post-vaccination.

The other three vaccinates were separated from these challenged animals for 24 hours and re-united thereafter. 28 days after challenge, all pigs were removed and destroyed.

vABV437 served as a positive control. A challenge control was included for 14 days starting at the moment of challenge in order to control challenge efficacy with LV-tH and SDSU#73, Animals were treated as described for the other animals during the challenge phase.

The allocation of the pigs is outlined in Table 1.

TABLE 1
Allocation of pigs to designated groups. Each mutant group
consisted of 5 vaccinated pigs and 1 sentinel (*so each
PRRSV-mutant had two groups). Groups 11 and 12 served
as challenge control groups (**) consisting of 5 animals
per group; only two of these pigs were intranasally exposed
to LV-tH or SDSU#73. All mutant groups were housed in
isolation recombinant facilities, whereas the wild type
groups were housed in standard isolation facilities.

| | | | N | |
|---|---|---|---|---|
| Group | Challenge | Vaccination | animals | Stables |
| 1 + 2 | LV-tH/ | 707 | 12* | 2 (geb. 46) |
| | SDSU#73 | | | |
| 3 + 4 | LV-tH/ | 741 | 12* | 2 (HRW- |
| | SDSU#73 | | | 223.030/40) |
| 5 + 6 | LV-tH/ | 746 | 12* | 2 (HRW- |
| | SDSU#73 | | | 223.050/60) |
| 7 + 8 | LV-tH/ | 688 | 12* | 2 (HRW- |
| | SDSU#73 | | | 223.070/80) |
| 9 + 10 | LV-tH/ | 437 | 12* | 2 (EHW) |
| | SDSU#73 | | | |
| 11 + 12 | LV-tH/ | | 10** | 2 (EHW) |
| | SDSU#73 | | | |

The vaccines were administered intramuscularly according to a SOP (2 ml deep intramuscularly in the neck halfway between the shoulder and the right ear; min titer 10⁵ TCID₅₀/ml). All inoculae were titrated before and after usage and were stored on melting ice at all times.

Experimental Animals

70 SPF pigs of 8-weeks old, tested free of PRRSV.

Execution of the Study (Table 2)

TABLE 2
Course of the study valid for each of the mutant groups.

| Day | Action |
|---|---|
| −5 till 0 | Acclimatisation of animals |
| −2 | Serum sampling for IDEXX-ELISA |
| Daily | General clinical status |
| 0 | Vaccination of 5 animals per group (2 ml intramuscular) |
| 1 | Sentinels |
| 3 × per week | Serum sampling for virus isolation (3 × per week) and |
| sampling | IDEXX-ELISA |
| | (1 × week) |
| Dag 28 | Removal of sentinels and challenge of 2 vaccinates with |
| | LV-tH or US virus (in stable 1 and 2 per mutant group, |
| | respectively) |
| 3 × per week | Serum sampling for virus isolation (3 × per week) and |
| sampling | IDEXX-ELISA |
| | (1 × week) |
| 56 | Finalization; destruction of pigs |

### Results

No adverse reactions were noted after exposure of the mutant virus or wild-type viruses to the pigs in each of the groups.

Tables 3 and 4 show the results of the PRRS virus isolation from serum and calculated viraemia scores. Incidences of viraemia at defined sampling points were determined by virus isolation on porcine alveolar macrophages using routine and published techniques; Virus positivity at a serum sample dilution of 1:10 was designated (+), and (++) means virus positivity at a serum sample dilution of 1:100. These results were used to calculate a group total “viraemia score” as (type 1) the percentage of the virus-exposed animals in each group (each virus positive animal at each time-point=1 point, so a max score of 100% (=12/12) can be obtained, and (type 2) as the percentage of maximal viraemia of the exposed animals. In the latter case, a max score of 100% (=24/24) can be obtained based upon the fact that max viraemia is scored as 2 points (1:100 dilution of the samples) for each individual animal. All mutant virus groups showed a reduced type 1 and type 2 viremia score as compared to vABV437. vABV707 vaccinated pigs showed a reduced type 1 and type 2 viraemia score prior to challenge as compared to the score of the pigs in all other groups. At the moment of challenge no animals were shown to be viraemic any more. All sentinels became viraemic and sero-converted, meaning that the viruses shedded from the exposed pigs to the sentinels. It is shown that primary exposure of the mutant viruses to the pigs renders an effective immunological response as determined by a near complete prevention of viraemia after homologous wild-type challenge and a firm reduction of viraemia after heteroogous challenge as compared to challenge controls. Vaccinated sentinels were effectively protected. No differences could be documented in serological responses after vaccination and challenge between each of the groups studied.

Challenge controls all show viraemia during the course of the 14-day study, where the viraemia is most predominant in the intranasally exposed pigs.

TABLE 3
Type 1 viraemia score. A group total “viraemia score” was
calculated as the percentage of the virus-exposed animals in each
group. Each virus positive animal at each time-point = 1 point,
so a max score of 100% (=12/12) can be obtained.

| | | | | | | Wild- |
|---|---|---|---|---|---|---|
| dpi | vABV707 | vABV741 | vABV746 | vABV688 | vABV437 | type |
| 0 | 0.0 | 0.0 | 0.0 | 0.0 | 0.0 | |
| 2 | 0.0 | 8.3 | 25.0 | 16.7 | 75.0 | |
| 4 | 16.7 | 83.3 | 91.7 | 75.0 | 100.0 | |
| 7 | 91.7 | 83.3 | 91.7 | 100.0 | 100.0 | |
| 9 | 91.7 | 91.7 | 91.7 | 83.3 | 100.0 | |
| 11 | 50.0 | 100.0 | 66.7 | 100.0 | 100.0 | |
| 14 | 66.7 | 83.3 | 83.3 | 83.3 | 100.0 | |
| 16 | 33.3 | 58.3 | 58.3 | 66.7 | 75.0 | |
| 18 | 41.7 | 16.7 | 25.0 | 33.3 | 50.0 | |
| 21 | 25.0 | 8.3 | 33.3 | 16.7 | 91.7 | |
| 23 | 25.0 | 16.7 | 25.0 | 0.0 | 41.7 | |
| 25 | 8.3 | 0.0 | 0.0 | 16.7 | 16.7 | |
| 28 | 0.0 | 0.0 | 0.0 | 0.0 | 0.0 | 0 |
| 30 | 10.0 | 0.0 | 30.0 | 30.0 | 10.0 | 0 |
| 32 | 20.0 | 0.0 | 10.0 | 20.0 | 40.0 | 40 |
| 35 | 20.0 | 10.0 | 10.0 | 20.0 | 20.0 | 60 |
| 37 | 0.0 | 30.0 | 0.0 | 20.0 | 20.0 | 90 |
| 39 | 10.0 | 0.0 | 0.0 | 0.0 | 30.0 | 90 |
| 42 | 0.0 | 0.0 | 0.0 | 0.0 | 10.0 | 100 |
| 44 | 0.0 | 0.0 | 0.0 | 0.0 | 0.0 | |
| 46 | 0.0 | 0.0 | 0.0 | 0.0 | 0.0 | |
| 49 | 0.0 | 0.0 | 0.0 | 0.0 | 0.0 | |
| 51 | 0.0 | 0.0 | 0.0 | 0.0 | 0.0 | |
| 53 | 0.0 | 0.0 | 0.0 | 0.0 | 0.0 | |
| 56 | 0.0 | 0.0 | 0.0 | 0.0 | 0.0 | |

TABLE 4
Type 2 viraemia score, calculated as the percentage of maximal
viraemia of the exposed animals. A max score of 100% (=24/24)
can be obtained based upon the fact that max viraemia is scored as
2 points (1:100 dilution of the samples) for each individual
animal at each time point.

| | | | | | | Wild- |
|---|---|---|---|---|---|---|
| dpi | vABV707 | vABV741 | vABV746 | vABV688 | vABV437 | type |
| 0 | 0.0 | 0.0 | 0.0 | 0.0 | 0.0 | |
| 2 | 0.0 | 4.2 | 12.5 | 8.3 | 37.5 | |
| 4 | 8.3 | 50.0 | 54.2 | 50.0 | 70.8 | |
| 7 | 45.8 | 58.3 | 62.5 | 66.7 | 83.3 | |
| 9 | 54.2 | 50.0 | 45.8 | 50.0 | 58.3 | |
| 11 | 25.0 | 70.8 | 37.5 | 54.2 | 95.8 | |
| 14 | 33.3 | 62.5 | 41.7 | 45.8 | 70.8 | |
| 16 | 16.7 | 45.8 | 33.3 | 33.3 | 41.7 | |
| 18 | 20.8 | 8.3 | 12.5 | 16.7 | 37.5 | |
| 21 | 12.5 | 8.3 | 16.7 | 8.3 | 50.0 | |
| 23 | 12.5 | 8.3 | 8.3 | 0.0 | 41.7 | |
| 25 | 4.2 | 0.0 | 0.0 | 8.3 | 8.3 | |
| 28 | 0.0 | 0.0 | 0.0 | 0.0 | 0.0 | 0 |
| 30 | 5.0 | 0.0 | 15.0 | 15.0 | 5.0 | 0 |
| 32 | 10.0 | 0.0 | 5.0 | 10.0 | 20.0 | 40 |
| 35 | 10.0 | 5.0 | 5.0 | 10.0 | 10.0 | 60 |
| 37 | 0.0 | 15.0 | 0.0 | 10.0 | 10.0 | 90 |
| 39 | 5.0 | 0.0 | 0.0 | 0.0 | 15.0 | 90 |
| 42 | 0.0 | 0.0 | 0.0 | 0.0 | 5.0 | 100 |
| 44 | 0.0 | 0.0 | 0.0 | 0.0 | 0.0 | |
| 46 | 0.0 | 0.0 | 0.0 | 0.0 | 0.0 | |
| 49 | 0.0 | 0.0 | 0.0 | 0.0 | 0.0 | |
| 51 | 0.0 | 0.0 | 0.0 | 0.0 | 0.0 | |
| 53 | 0.0 | 0.0 | 0.0 | 0.0 | 0.0 | |
| 56 | 0.0 | 0.0 | 0.0 | 0.0 | 0.0 | |

Conclusion

The studied recombinant mutant PRRS viruses show a reduced virulence as determined by a reduction of viraemia (length and height) as compared to wild-type (vABv437). All mutants instigate an effective immune response for the protection of pigs against a wild-type field PRRSV. The homologous protection seems to be somewhat more effective than the heterologous one. The humoral response is measurable by a commercial ELISA (IDEXX) in all cases. No adverse reactions are elicited.

1. An Arterivirus having at least some of its original arteriviral nucleic acid encoding ORF-7 deleted.

2. A replicon according to claim 1 capable of in vivo RNA replication.

3. A replicon according to claim 2, further comprising a nucleic acid derived from at least one heterologous micro-organism.

4. A replicon according to claim 1, wherein said replicon comprises an RNA transcript of an infectious cDNA.

5. A replicon according to claim 1, wherein said replicon comprises a functional kissing loop interaction.

6. A replicon according to claim 1, wherein said arteriviral nucleic acid encoding ORF-7 produces a C-terminally truncated ORF-7 polypeptide.

7. A replicon according to claim 6 wherein said truncation does not effect the production of viable virus.

8. A replicon according to claim wherein said Arterivirus comprises a porcine reproductive and respiratory syndrome virus (PRRSV).

9. A replicon according to claim 8 comprising a nucleic acid modification leading to an at most 6 amino acid truncation of ORF-7.

10. A replicon according to claim 8 comprising a nucleic acid modification of a 34-nucleotided stretch of ORF-7 from position 14653-14686 and a nucleic acid modification of the 3′-UTR from position 14996-15034.

11. A replicon according to claim 3, wherein said heterologous micro-organism comprises a pathogen.

12. A replicon according to claim 11 wherein said pathogen is a virus.

13. A method of using a replicon according to of claim 2 to obtain a vaccine.

14 and 15 (Canceled)

16. A vaccine comprising:
an Arterivirus nucleic acid, wherein said Arterivirus nucleic acid comprises a functional kissing loop interaction.

17. The vaccine of claim 15, further comprising a deletion in an ORF-7 polypeptide.

18. A method of vaccinating an animal, the method comprising:
administering a vaccine according to claim 15 to an animal.

19. The method according to claim 17, wherein the animal is a swine.
